(19) United States
(12) Patent Application Publication
(10) Pub. No.: US 2016/0361476 A1
(43) Pub. Date: Dec. 15, 2016

(54) SYSTEM FOR TISSUE MANIPULATION

(71) Applicant: BDBC SCIENCES CORP., Richmond (CA)

(72) Inventor: Lotien R. Huang, Chestnut Hill, MA (US)

(21) Appl. No.: 15/120,833

(22) PCT Filed: Feb. 27, 2015
(86) PCT No.: PCT/US15/18080

§ 371 Date: Aug. 23, 2016

Related U.S. Application Data
(60) Provisional application No. 61/946,365, filed on Feb. 28, 2014.

(51) Int. Cl. A61M 1/00

(52) U.S. Cl. CPC A61M 1/0056; A61M 2202/08; A61M 2205/36; A61M 2205/6018

(57) ABSTRACT

A system for manipulation of a tissue sample includes a chassis, a chamber defined in the chassis and configured to receive and retain a complimentary device including a sample processing compartment disposed between sheets of a flexible material and a waste chamber disposed between the sheets of the flexible material. The complimentary device is configured to retain the tissue sample during manipulation of the tissue sample by the system. A fluid mixing sub-system is configured to agitate and mix a fluid including the tissue sample within the sample processing compartment. A temperature control sub-system including at least one of a first heating element and a first cooling element is configured and arranged to be in thermal communication with the sample processing compartment. An electronic controller is in communication with, and programmed to control operation of the fluid mixing sub-system and the temperature control sub-systems.

### BACKGROUND

Autologous lipotransfer, often known as fat transfer, is a technique used in plastic surgery and cosmetic procedures where a patient's own fat tissue is harvested from one part of the body, typically the abdomen or thighs, and transferred to another part of the patient's body for cosmetic or therapeutic purposes. Autologous lipotransfer is often used in breast augmentation and reconstruction, facial rejuvenation, augmentation of the posterior, also known as the “Brazilian butt lift,” and other procedures.

Autologous lipotransfer typically involves three steps: liposuction, fat processing, and reinjection.

Liposuction is a procedure where fat tissue from a patient's body is removed and optionally harvested using suction. It is also referred to as liposculpture, lipoplasty, and suction-assisted lipectomy. During liposuction, a small tube called a cannula is inserted through tiny cuts in the skin. Fat tissue is suctioned out through the cannula as the doctor moves it around under the skin to target specific fat deposits. Liposuction may be performed entirely manually, using a cannula attached to a syringe, or may be performed with the assistance of a machine, which produces a vacuum and which provides a container to receive the fat tissue.

There are many variations and trademarks for various liposuction techniques, including a dry technique, a wet technique, a super wet technique, tumescent liposuction, ultrasound-assisted liposuction, power-assisted liposuction, waterjet-assisted liposuction, and VASER® liposuction, also referred to as LIPOSELECTION®, SMARTLIPO®, CoolLipo™, ProLipo PLUS™, LIPOLITE® laser liposuction technique, LipoTherme™, LipoControl™, etc.

Tumescent liposuction is a technique where an anesthetic solution containing lidocaine and epinephrine is injected into the fatty tissue of the patient before suction is applied. This technique has allowed liposuction to be performed with the patient under local anesthesia while minimizing blood loss and reducing the need and risks of general anesthesia. In some cases local anesthesia is used, and the patient may or may not be given a sedative to help relax. If a large area or volume of fat is being treated, general anesthesia or deep sedation with a local anesthetic may be used.

Power assisted liposuction (PAL) uses a powered device which provides a rapid in-and-out movement or a spinning rotation of an attached liposuction cannula driven by an electric motor or compressed air.

Ultrasound-assisted liposuction uses ultrasound to liquefy the fat, which makes it easier to remove. This technique may be particularly helpful in removing fat from the upper abdomen, sides, and back.

Waterjet-assisted liposuction relies on the power of a highly focused water jet to dislodge and remove fat from the body. The power of the water jet detaches fat cells and tissues from their surrounding tissues, allowing the suction cannula to move freely. This technique may have the benefit of reducing the possibility of trauma to surrounding tissues, including skin, muscles, nerves, blood vessels, and septal attachments.

Laser liposuction requires the use of tumescent fluid and uses a micro-cannula inserted through a small incision to deliver laser energy and heat into fat to facilitate fat removal.

### SUMMARY

Fat tissue harvested from a patient using liposuction is referred to as lipoaspirate. Lipoaspirate samples may be processed before reinjection. One type of fat processing includes a tissue wash step, where lipoaspirate is rinsed and washed to reduce the amount of residual blood and tumescent anesthesia in the lipoaspirate and provide a relatively pure graft for reinjection. Washing may include centrifugation, for example, in centrifugation tubes or in syringes, where lipoaspirate is separated into an aqueous phase layer containing blood and tumescent solution, an adipose tissue (fat tissue) layer, and a free oil layer under centrifugal force. The free oil layer may comprise oil from broken adipose cells. The adipose tissue layer may be harvested. Because of the large density difference in fat tissue and the aqueous solutions containing blood, fat washing may also be performed using settling under gravity. Alternatively, washing of lipoaspirate may be achieved using a filter mesh or a strainer, through which the aqueous solutions and free oil contained in the lipoaspirate are drained, and by which the fat tissue is retained.

Another type of processing may include extracting non-adipose cells from a fat tissue, for example, a lipoaspirate. Enzymes, such as collagenase, may be used to dissociate the tissue and release a heterogeneous cell population, including adipose cells, adipose derived stem cells, fibroblasts, endothelial progenitor cells, and pericytes. The adipose cell population may then be at least partially removed using settling, centrifugation, and/or filtering to produce a substantially non-fat cell population, which is often referred to as the stromal vascular fraction (SVF).

Stromal vascular fractions may be used as grafts for reinjection into the patient, or may be cultured to produce a larger population and/or a more homogeneous population of cells, for example, adipose derived stem cells, progenitor cells, fibroblasts, or fibroblast-like cells. The cell culturing and expansion steps may rely on the adherence property of the cells to a surface of a culturing container, for example, a flask, a petri dish, etc. The stromal vascular fraction may contain cells that have the potential to differentiate along multiple lineages, for example, osteogenic adipogenic and chondrogenic lineages. The SVF cells may be used to derive chondrocyte, osteocytes, adipocytes, many different cell types, blood vessels, and even tissues. The stromal vascular fraction may further contain cells that can be induced to become pluripotent stem cells, from which many other cell types, or even tissues, may be derived, engineered, or used for therapies.

Cells derived from adipose tissue could potentially provide a source of cells for tissue engineering to make, for example, cartilage, bone, and fat tissues, among many other potential tissue types. Cells derived from lipoaspirate, such as the stromal vascular fraction and adipose derived stem cells, may be used in cosmetic procedures such as facial rejuvenation, wrinkle reduction, breast augmentation, as well as in clinical trials testing procedures for treating diseases, conditions, and indications including wounds and injuries, viral diseases, urinary tract, sexual organ, and pregnancy conditions, general pathology symptoms, skin and connective tissue diseases, respiratory tract (including lung and bronchial) diseases, nutritional and metabolic diseases, nervous system diseases, muscle, bone, and cartilage diseases, mouth and tooth diseases, immune system diseases, heart and blood diseases, gland and hormone related diseases, eye diseases, birth defects and abnormalities, digestive system diseases, cancers and neoplasms, blood and lymph conditions, etc.

Yet another type of processing may include preparing a tissue sample for cryopreservation, and cryopreserving the tissue. Cryopreservation of tissue or cell samples is often times referred to as tissue or cell banking, respectively. In tissue or cell banking, a sample or specimen is treated with a cryoprotectant, a substance that is used to protect biological tissue from freezing damage (i.e. that due to ice formation). An example of commonly used cryoprotectant is dimethyl sulfoxide (DMSO). Glycols (alcohols containing at least two hydroxyl groups), such as ethylene glycol, propylene glycol, and glycerol have also been used as cryoprotectants for research purposes. Glycerol and DMSO may be used by cryobiologists to reduce ice formation in sperm and embryos that are cryopreserved in liquid nitrogen. The preserved cells and tissues may later be re-injected into the same patient, used as a source of stem cells or progenitor cells, used to produce pluripotent stem cells, or used for other purposes.

Re-injection of lipoaspirate may involve manual re-injection using a syringe. For large volume lipotransfer, manual re-injection may be very time consuming, labor intensive, and may render a patient prone to contamination and infection. In many previously known procedures, fat grafts, tissue sample materials, and/or cellular materials are manipulated in an open environment, which subjects the materials to contamination and, in turn, puts the patients at risk for infection and puts the operators at risk for contracting infectious diseases. In other previously known procedures, manipulating the tissue samples involves manual manipulation of several closed systems or semi-closed systems that are not integrated. However, manual manipulations still require trained personnel often involving sterile and non-sterile personnel in an operating room setting, lengthen the time to perform the procedures, are subject to operator error, and cause process variations.

The inventor of the present disclosure has determined that it may be desirable to provide a tool including a closed system which may enable safe, standardized, efficient, and minimal contamination risk procedures of lipotransfer, sample manipulation or tissue processing. The inventor of the present disclosure has also determined that it may be desirable to provide a method for performing fat transfer and/or tissue processing using a power assisted, semi-automated, or automated tool. Aspects and embodiments disclosed herein may address one or both of these needs.

In one embodiment of the present disclosure, a system comprises a closed or semi-closed complementary device and a tissue manipulation machine. The system may perform tissue washing, dissociation, and cell filtration.

In another embodiment of the present disclosure, a tissue manipulation machine automates tissue washing, dissociation, and cell filtration procedures in a closed system comprising a complementary device.

In yet another embodiment of the present disclosure, a system comprises a complementary device and a tissue manipulation machine wherein the system washes a lipoaspirate sample, enzymatically digests the lipoaspirate sample to release a released cell population, removes debris and adipose cells from the released cell population, and optionally brings the released cell population in contact with a serum solution in an automated manner following a pre-programmed protocol.

In yet another embodiment of the present disclosure, a system for preparing a tissue sample for cryopreservation comprises a complementary device and a tissue manipulation machine. The system may perform several steps including sampling a portion of the tissue sample for a test, washing the tissue sample, and mixing the tissue sample with a cryoprotectant using a pre-programmed protocol.

In yet another embodiment of the present disclosure, a system comprises a complementary device and a tissue manipulation machine. The complementary device may be sterile, single use, and may include an RFID tag containing an instruction. The tissue manipulation machine may contain a controller, for example, a programmable computer, an RFID reader, and a plurality of software protocols. The tissue manipulation machine may read the instruction from the complementary device using the RFID reader and execute a software protocol based on the instruction.

In yet another embodiment of the present disclosure, a system comprises a programmable tissue manipulation machine including a plurality of pre-programmed processes, and a complementary device including an RFID tag. A pre-programmed process is automatically selected, activated, and executed according to the information contained in the RFID tag.

In yet another embodiment of the present disclosure, a system comprises a tissue manipulation machine, a plurality of software protocols, and a single-use complementary device. The tissue manipulation machine contains an RFID reader. The complementary device includes an RFID tag. A pre-programmed process is uniquely selected, activated, and executed on the complementary device by a controller of the programmable tissue manipulation machine according to the information contained in the RFID tag.

In yet another embodiment of the present disclosure, a system for power assisted lipotransfer comprises a complementary device and a tissue manipulation machine. The complementary device provides a sterile environment to accommodate the suctioned lipoaspirate. The tissue manipulation machine provides suction through a tissue extraction device, for example, a cannula for liposuction, power to move the lipoaspirate out of an injection device, for example, a cannula, for reinjection, and a fluid to rinse the lipoaspirate, thereby facilitating liposuction, fat washing, and fat injection in a sterile and semi-closed or closed system.

In yet another embodiment of the present disclosure, a system configured to perform liposuction, fat washing, and fat injection in a sterile and semi-closed or closed device is used to perform liposuction on a patient. The lipoaspirate from the patient may be washed within the system and re-injected into the patient using power provided by the system, thereby performing a power assisted lipotransfer procedure.

In yet another embodiment of the present disclosure, a liposuction procedure is performed on a patient using a device configured to perform liposuction, fat washing, and fat injection in a sterile and semi-closed or closed system. Fat tissue (lipoaspirate) is removed from one part of the patient and collected in the device using a vacuum provided by the device. The lipoaspirate is then optionally washed in the device using a solution, and transferred back into another part of the patient using the same device, which drives the lipoaspirate during the transfer.

In yet another embodiment of the present disclosure, a method for lipotransfer comprises performing a liposuction procedure on a patient using a multifunctional tool comprising a complementary device. The fat tissue procured from the liposuction procedure is collected and then washed in the complementary device. The washed fat tissue is then re-injected into the patient using the multifunctional tool.

In yet another embodiment of the present disclosure, a liposuction procedure is performed on a patient at a clinical site to collect a fat tissue sample. The fat tissue sample is then processed at the clinical site within a short period of time, for example, within about 30 min, to prepare the tissue sample for cryopreservation. The tissue sample is then cooled at the clinical site within a short period of time, for example, within about 90 minutes, to cryopreserve the tissue sample.

In yet another embodiment of the present disclosure, a procedure is performed on a patient at a clinical site to collect a tissue sample. The tissue sample is processed immediately to produce a cell population. The cell population is then immediately mixed with a cryoprotectant and cooled to below −20° C., thereby preserving the maximum viability and functions of the cell population. The procedure may be a liposuction procedure and the tissue sample may be a lipoaspirate sample.

In accordance with an aspect of the present disclosure, there is provided a system for manipulation of a tissue sample. The system comprises a chassis, a chamber defined in the chassis and configured to receive and retain a complimentary device including a sample processing compartment disposed between sheets of a flexible material and a waste chamber disposed between the sheets of the flexible material and selectively fluidly connected to an outlet of the sample processing compartment. The complimentary device is configured to retain the tissue sample during manipulation of the tissue sample by the system. A fluid mixing sub-system is disposed in the chamber and configured to agitate and mix a fluid including the tissue sample within the sample processing compartment. A temperature control sub-system includes at least one of a first heating element and a first cooling element disposed in the chamber and configured and arranged to be in thermal communication with the sample processing compartment. An electronic controller in communication with, and programmed to control operation of, the fluid mixing sub-system and the temperature control sub-system.

In some embodiments, the system further comprises a fluid control sub-system disposed in the chassis and controlled by the electronic controller and a user interface in communication with the electronic controller.

In some embodiments, the temperature control sub-system includes one of second a heating element and a second cooling element disposed in the chassis and in thermal communication with a rinse solution disposed within a source of rinse solution.

In some embodiments, the fluid control sub-system includes a valve actuator configured to mechanically manipulate a valve disposed in the complimentary device, the valve having a state providing for gravity drain of a fluid from the sample processing compartment into the waste chamber.

In some embodiments, the fluid control sub-system further includes a first pump configured to withdraw a rinse solution from a source of rinse solution and direct the rinse solution into the sample processing compartment.

In some embodiments, the first pump comprises a first syringe and the fluid control sub-system further includes a first linear actuator configured to manipulate a plunger of the first syringe.

In some embodiments, the fluid control sub-system further includes a second pump configured to direct a treatment solution into the sample processing compartment.

In some embodiments, the second pump comprises a second syringe and the fluid control sub-system further includes a second linear actuator configured to manipulate a plunger of the second syringe.

In some embodiments, the system further comprises a third syringe configured to withdraw treated cells from the complimentary device.

In some embodiments, the fluid control sub-system further includes a third linear actuator configured to manipulate a plunger of the third syringe.

In some embodiments, the fluid control system further includes a sensor in communication with the electronic controller, the sensor being configured to monitor one of a flow rate and a property of a fluid in the system selected from a color of the tissue sample and a turbidity of the tissue sample.

In some embodiments, the fluid mixing sub-system includes a roller configured to agitate and mix fluid within the sample processing compartment.

In some embodiments, the fluid mixing sub-system includes a rotating arm configured to agitate and mix fluid within the sample processing compartment.

In some embodiments, the fluid mixing sub-system includes a moving plate configured to agitate and mix fluid within the sample processing compartment.

In some embodiments, the system further comprises a detection feedback system including a sensor in communication with the electronic controller. The sensor configured and arranged to provide an indication of a weight of a bag of a rinsing solution disposed on a platform coupled to the chassis. The fluid control sub-system is configured to dispense a volume of rinsing solution into the sample processing compartment determined by a change in weight of the bag.

In some embodiments, the system further comprises a detection feedback system including a sensor in communication with the electronic controller. The sensor configured and arranged to one of provide an indication of whether the complimentary device is properly mounted within the chamber, provide an indication of whether a syringe is properly mounted on the system, provide an indication of whether a door of the chamber is closed, and provide an indication of whether the door of the chamber is locked.

In some embodiments, the system further comprises an identification tag reader configured to read an identification tag included on the complimentary device.

In some embodiments, the controller is configured to execute a tissue manipulation protocol defined by information read from the identification tag by the identification tag reader.

In accordance with another aspect, there is provided a method of processing a tissue sample. The method comprises introducing the tissue sample into a sample processing compartment of a device including the sample processing compartment and a waste chamber, the sample processing compartment and the waste chamber being disposed between common sheets of a flexible material, the waste chamber being selectively fluidly connected to an outlet of the sample processing chamber, mounting the device within a processing chamber of a tissue manipulation apparatus, agitating and mixing the tissue sample within the sample processing compartment with a fluid mixing sub-system disposed in the processing chamber under control of an electronic controller of the tissue manipulation apparatus, and one of heating and cooling the tissue sample with a temperature control sub-system including at least one of a first heating element and a first cooling element disposed in the processing chamber and in thermal communication with the sample processing compartment under control of the electronic controller.

In some embodiments, the method further comprises washing the tissue sample in the sample processing compartment by dispensing a measured volume of a rinsing solution into the sample processing compartment under control of the electronic controller.

In some embodiments, the method further comprises digesting the tissue sample in the sample processing compartment by dispensing a measured volume of a dissociation solution into the sample processing compartment under control of the electronic controller.

In some embodiments, the method further comprises mechanically manipulating a valve in fluid communication between the sample processing compartment and the waste chamber under control of the electronic controller, and mechanically manipulating the valve causing waste fluid to flow under the influence of gravity from the sample processing compartment to the waste chamber.

In some embodiments, the method further comprises preparing the tissue sample for cryopreservation by dispensing a measured volume of a cryoprotectant into the sample processing compartment under control of the electronic controller.

In some embodiments, the method further comprises withdrawing a treated tissue sample from the device under control of the electronic controller. In accordance with another aspect, there is provided a system for tissue manipulation. The system comprises a tissue processing unit, a first cannula connector fluidly connected to the tissue processing unit, a collection canister disposed within the tissue processing unit, a mesh chamber including a mesh filter disposed within the collection canister, and a vacuum source in communication with the tissue processing unit.

In some embodiments, the system further comprises a tissue pump fluidly connected between the cannula connector and the tissue processing unit.

In some embodiments, the system further comprises a source of rinse solution in fluid communication with the collection canister.

In some embodiments, the system is configured to withdraw adipose tissue from a patient into the collection canister.

In some embodiments, the system is further configured to reinject the adipose tissue into the patient.

In some embodiments, the system is further configured to rinse the adipose tissue prior to reinjecting the adipose tissue into the patient.

In some embodiments, the system further comprises a second cannula connector for reinjecting the adipose tissue into the patient.

In some embodiments, the system further comprises a vent valve and vent filter in communication with an internal volume of the tissue processing unit.

In some embodiments, the system further comprises a fluid waste collection chamber.

In accordance with another aspect, there is provided a method of operating a system for tissue manipulation to process a tissue sample. The system comprises a tissue processing unit, a first cannula connector fluidly connected to the tissue processing unit, a collection canister disposed within the tissue processing unit, a mesh chamber including a mesh filter disposed within the collection canister, and a vacuum source in communication with the tissue processing unit.

In some embodiments, processing the tissue sample includes washing the tissue sample.

In some embodiments, processing the tissue sample includes dissociating the tissue.

In some embodiments, processing the tissue sample includes preparing the tissue sample for cryopreservation.

In accordance with another aspect, there is provided a method of performing a procedure on a patient with a system for tissue manipulation. The system comprises a tissue processing unit, a first cannula connector fluidly connected to the tissue processing unit, a collection canister disposed within the tissue processing unit, a mesh chamber including a mesh filter disposed within the collection canister, and a vacuum source in communication with the tissue processing unit.

In some embodiments, the procedure includes liposuction.

In some embodiments, the procedure includes lipotransfer.

In some embodiments, the procedure includes autologous lipotransfer.

In some embodiments, the procedure includes fat injection.

In accordance with another aspect, there is provided a system for manipulation of a tissue sample. The system comprises a chassis and a chamber defined in the chassis and configured to receive and retain a complementary device including a flexible sample processing compartment selectively fluidly connected to a source of a first solution, and a waste chamber selectively fluidly connected to an outlet of the sample processing compartment, the complementary device configured to retain the tissue sample and receive the first solution during manipulation of the tissue sample by the system. A fluid mixing sub-system is disposed in the chamber and configured to agitate and mix a fluid including the first solution and the tissue sample within the sample processing compartment. A temperature control sub-system includes at least one of a first heating element and a first cooling element configured and arranged to be in thermal communication with the sample processing compartment. An electronic controller is in communication with, and programmed to control operation of, the fluid mixing sub-system and the temperature control sub-system.

In some embodiments, the system further comprises a fluid control sub-system disposed in the chassis and controlled by the electronic controller and a user interface in communication with the electronic controller.

In some embodiments, the waste chamber sample and the processing compartment are disposed between sheets of a flexible material.

In some embodiments, the sample processing compartment and the waste chamber are disposed between common sheets of a flexible material.

In some embodiments, the fluid mixing sub-system is configured to manipulate at least a part of the flexible sample processing compartment, providing massaging action to the flexible sample processing compartment.

In some embodiments, the fluid control sub-system includes a valve actuator configured to mechanically manipulate a valve disposed in the complementary device, the valve having a state providing for gravity drain of a fluid from the sample processing compartment into the waste chamber.

In some embodiments, the fluid control sub-system further includes a first pump configured to withdraw the first solution and direct the first solution into the sample processing compartment. The first pump may comprise a first syringe included in the complementary device and the fluid control sub-system further includes a first linear actuator configured to manipulate a plunger of the first syringe. The system may further include a second pump configured to direct a second solution into the sample processing compartment. The second pump may comprise a second syringe included in the complementary device and the fluid control sub-system further includes a second linear actuator configured to manipulate a plunger of the second syringe.

In some embodiments, the first solution is a rinse solution and the second solution is a reagent solution comprising enzyme.

In some embodiments, the system further comprises a third syringe configured to withdraw treated cells from the complementary device. The fluid control sub-system may further include a third linear actuator configured to manipulate a plunger of the third syringe.

In some embodiments, the system further comprises a detection feedback system including a sensor in communication with the electronic controller, the sensor configured and arranged to one of provide an indication of whether the complementary device is properly mounted within the chamber, provide an indication of whether a syringe is properly mounted on the system, provide an indication of whether a door of the chamber is closed, and provide an indication of whether the door of the chamber is locked.

In some embodiments, the system further comprises a detection feedback system including a sensor in communication with the electronic controller, the sensor configured and arranged to provide an indication of a weight of a bag of a rinsing solution disposed on a platform coupled to the chassis, the fluid control sub-system configured to dispense an volume of rinsing solution into the sample processing compartment determined by a change in weight of the bag.

In some embodiments, the system further comprises an identification tag reader configured to read an identification tag included on the complementary device. The controller may be configured to execute a tissue manipulation protocol defined by information read from the identification tag by the identification tag reader.

In some embodiments, the temperature control sub-system is configured to be in thermal communication with the sample processing compartment using forced air.

In some embodiments, the temperature control sub-system includes a plate configured to be in thermal communication with the at least one of the first heating element and the first cooling element and in physical contact with the complementary device.

In some embodiments, the fluid mixing sub-system includes a roller configured to agitate and mix fluid within the sample processing compartment.

In some embodiments, the fluid mixing sub-system includes a rotating arm configured to agitate and mix fluid within the sample processing compartment.

In some embodiments, the fluid mixing sub-system includes a moving plate configured to agitate and mix fluid within the sample processing compartment.

In some embodiments, the complementary device further includes a filter configured to remove debris from treated cells.

In some embodiments, the sample processing compartment has a surface to volume ratio of greater than 3 cm⁻¹.

In some embodiments, the fluid control system further includes a sensor in communication with the electronic controller, the sensor configured to monitor one of a flow rate and a property of a fluid in the system selected from a color of the tissue sample and a turbidity of the tissue sample.

In some embodiments, the temperature control sub-system is configured to heat up the tissue in the sample processing compartment to 35° C. or greater within 2 minutes.

In accordance with another aspect, there is provided a method of processing a tissue sample. The method comprises mounting a device including a sample processing compartment disposed between sheets of a flexible material, and a waste chamber selectively fluidly connected to an outlet of the sample processing chamber, onto a processing chamber of a tissue manipulation apparatus, introducing the tissue sample into the sample processing compartment of the device, and introducing a fluid into the sample processing compartment to treat the tissue. The method further comprises agitating and mixing the tissue sample within the sample processing compartment with a fluid mixing sub-system disposed at the processing chamber under control of an electronic controller of the tissue manipulation apparatus, and one of heating and cooling the tissue sample with a temperature control sub-system including at least one of a first heating element and a first cooling element disposed at the processing chamber and in thermal communication with the sample processing compartment under control of the electronic controller.

In some embodiments, the method further comprises washing the tissue sample in the sample processing compartment by dispensing a measured volume of a rinse solution into the sample processing compartment under control of the electronic controller.

In some embodiments, the method further comprises digesting the tissue sample in the sample processing compartment by dispensing a measured volume of a dissociation solution into the sample processing compartment under control of the electronic controller. The dissociation solution may contain an enzyme.

In some embodiments, the sample processing compartment and the waste chamber are disposed between common sheets of a flexible material.

In some embodiments, the method further comprises mechanically manipulating a valve in fluid communication between the sample processing compartment and the waste chamber under control of the electronic controller, mechanically manipulating the valve causing a waste fluid to flow under the influence of gravity from the sample processing compartment to the waste chamber.

In some embodiments, the method further comprises withdrawing a fluid containing cells from the device under control of the electronic controller.

In some embodiments, the method further comprises removing debris using a filter included in the device.

In some embodiments, the tissue is an adipose tissue having a weight, wherein the method further comprises digesting the tissue sample in the sample processing compartment by dispensing a measured volume of a dissociation solution comprising collagenase into the sample processing compartment under control of the electronic controller, and wherein the method further comprises collecting a fluid containing viable nucleated cells from the device.

In some embodiments, the number of viable nucleated cells collected from a unit weight of the adipose tissue is more than 700,000 per gram of adipose tissue.

In some embodiments, the intra-sample coefficient of variance of viable nucleated cells collected from a unit weight of the adipose tissue is no greater than 5%.

In some embodiments, the method is performed in a time of no longer than 55 minutes.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the disclosure. In the figures, each identical or nearly identical component that is illustrated in various figures is represented by a like numeral. For purposes of clarity, not every component may be labeled in every figure. In the figures:

FIG. 1A is an elevational view of an embodiment of a system for automated tissue manipulation;

FIG. 1B is another elevational view of a machine of the system of FIG. 1A;

FIG. 1C is a schematic view of components of the system of FIG. 1A;

FIG. 1D is an isometric view of the system of FIG. 1A;

FIG. 1E is another isometric view of a machine of the system of FIG. 1A;

FIG. 1F is a block diagram of a system for automated tissue manipulation;

FIG. 1G is another block diagram of a system for automated tissue manipulation;

FIG. 2A is a schematic diagram of another embodiment of a system for automated tissue manipulation;

FIG. 2B is a schematic diagram of another embodiment of a system for automated tissue manipulation;

FIG. 2C is a schematic diagram of another embodiment of a system for automated tissue manipulation;

FIG. 2D is a schematic diagram of another embodiment of a system for automated tissue manipulation;

FIG. 2E is a schematic diagram of another embodiment of a system for automated tissue manipulation;

FIG. 2F is a schematic diagram of another embodiment of a system for automated tissue manipulation;

FIG. 3A is a schematic view of a syringe pump;

FIG. 3B is a schematic view of another syringe pump;

FIG. 3C is a schematic view of yet another syringe pump;

FIG. 4A is a block diagram of another system for automated tissue manipulation;

FIG. 4B is another block diagram of another system for automated tissue manipulation;

FIG. 5 is a temperature profile measured on a system for automated tissue manipulation;

FIG. 6A is bar plot showing viable cell recovery data comparing a system for automated tissue manipulation with other systems known in the literature;

FIG. 6B is bar plot showing viable cell recovery data from three identical systems for automated tissue manipulation;

FIG. 7A is an image of three lipoaspirate bits drawn into three lines on a paper towel using a tissue manipulation system; and

FIG. 7B is a bar plot showing the weights of fourteen consecutive lipoaspirate bits dispensed using a tissue manipulation system.

### DETAILED DESCRIPTION

It should be appreciated that the present disclosure is not limited to processing adipose related tissue samples, such as an adipose tissue or a lipoaspirate sample. Many embodiments of the present disclosure are readily applicable to processing various tissue samples. The term “tissue sample” as used herein may include, but is not limited to, a tissue, a human tissue, an animal tissue, an epithelial tissue, a connective tissue, a nervous tissue, a muscle tissue, a solid tumor tissue, a polyp, a breast tissue, a uterus tissue, a tissue from an internal organ, a biopsy specimen, a placenta tissue, an umbilical cord tissue, a tissue containing stem cells, a pancreatic tissue, a brain tissue, a heart tissue, a heart muscle tissue, an adipose tissue, a lipoaspirate, a minced tissue, a minced adipose tissue, a melanoma tumor, a primary tumor, a secondary tumor, a foreskin, a skin tissue, a scalp tissue, a solid tissue, a tissue containing stroma, pancreatic islets, a pancreatic tissue, a liver tissue, a tissue containing progenitor cells and/or stem cells, a ligament tissue, a bone tissue, a mesenchymal tissue, a tissue containing cells of interest, a tissue containing hepatocytes, a tissue containing lymphocytes, a tissue containing T lymphocytes, a tumor containing T lymphocytes, a tumor containing tumor infiltrating lymphocytes, a tumor containing tumor reactive lymphocytes, a tissue containing leukocytes, a tissue containing fibroblasts, a tissue containing keratinocytes, a tissue containing chondrocytes, a tissue containing cardiomyocytes, a tissue containing oocytes, a tissue containing nerve cells, a retina tissue, an umbilical cord, a tissue from an umbilical cord, cells embedded in a matrix, cells embedded in an extracellular matrix, a tissue from a patient, plant tissues, a blood tissue, a bone marrow tissue, a cornea, a hair follicle, and other tissue pieces of biological origin, whether dead or alive. The term “tissue sample” as used herein may also include a multi cellular organism, a complete organism, algae, parasites, biomass, an aggregate of the above listed organisms, a food sample, hamburger patties, beef, lamb, chicken, pork, turkey, shellfish, fish, poultry, ground beef, ground meat, ground chicken, ground turkey, ground pork, ground lamb, hot dogs, corn dogs, mixed meat, candy bars, and/or peanut butter. The term “tissue sample” as used herein may also include an organ, for example, a heart, a brain, a liver, a kidney, a pancreas, a testicle, a breast, an ovary, an intestine, a stomach, a lung, a bladder, a penis, a colon, a gallbladder, a thymus, a gland, a tongue, an eye ball, an ear, a nose, a hand, a foot, an arm, a leg, a blood vessel, a minced sample of an organ, and any combination of the above listed samples. Many embodiments of the present disclosure are readily applicable to processing various tissue samples.

It should be appreciated that the present disclosure is not limited to plastic surgery, aesthetic medicine, or cosmetic applications. For example, one embodiment of the present disclosure includes a system that may be used for harvesting and preparing fat tissue for cryopreservation, also known as fat tissue banking. Another embodiment of the present disclosure includes a system for extracting cells from a tissue, for example, extracting adipose tissue derived cells from a lipoaspirate or extracting cells from a solid tumor, for cell banking, tissue banking, research, diagnosing diseases, stratifying patients, stratifying cancer patients to determine a course of treatment, molecular testing, extracting cells from a solid tumor for cell therapy, extracting immune cells, such as T lymphocytes, tumor infiltrating lymphocytes, and/or tumor reactive leukocytes, from a solid tumor for cell therapy, treating indications such as myocardial infarction, heart diseases, strokes, sports injuries, torn ligaments, bone fractures, burn wounds, wounds, non-healing wounds, ulcers, etc., and/or other clinical applications. Yet another embodiment of the present disclosure includes a system for extracting pathogen, for example bacteria, from a food specimen for food safety testing and monitoring.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

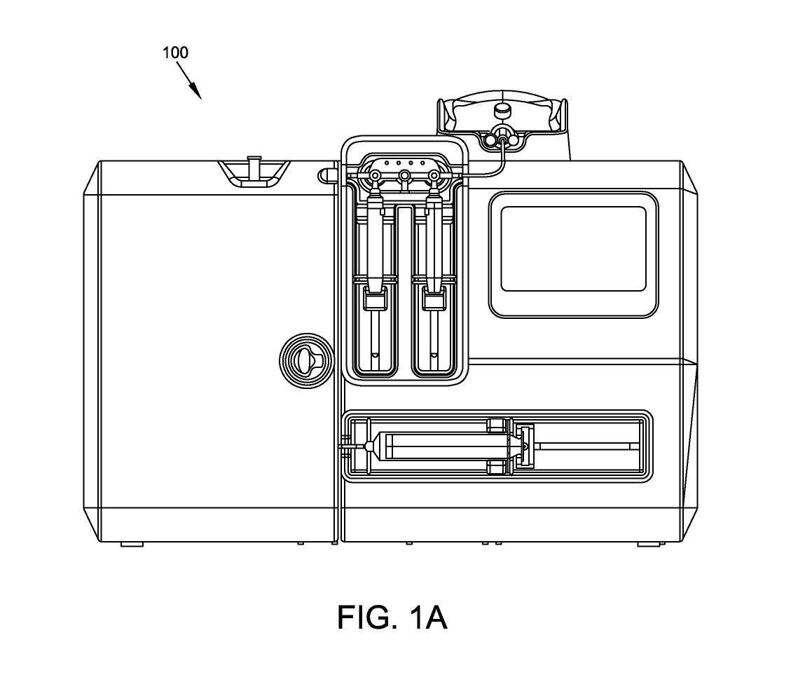

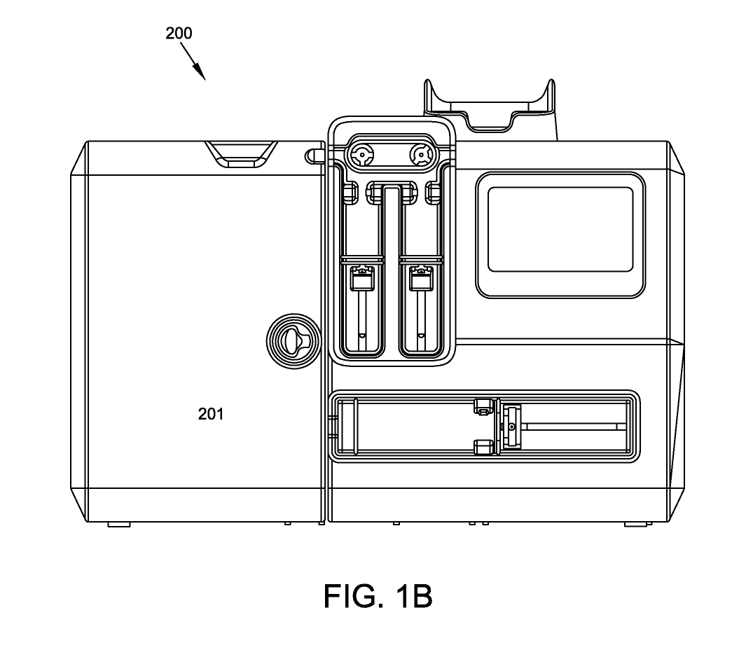

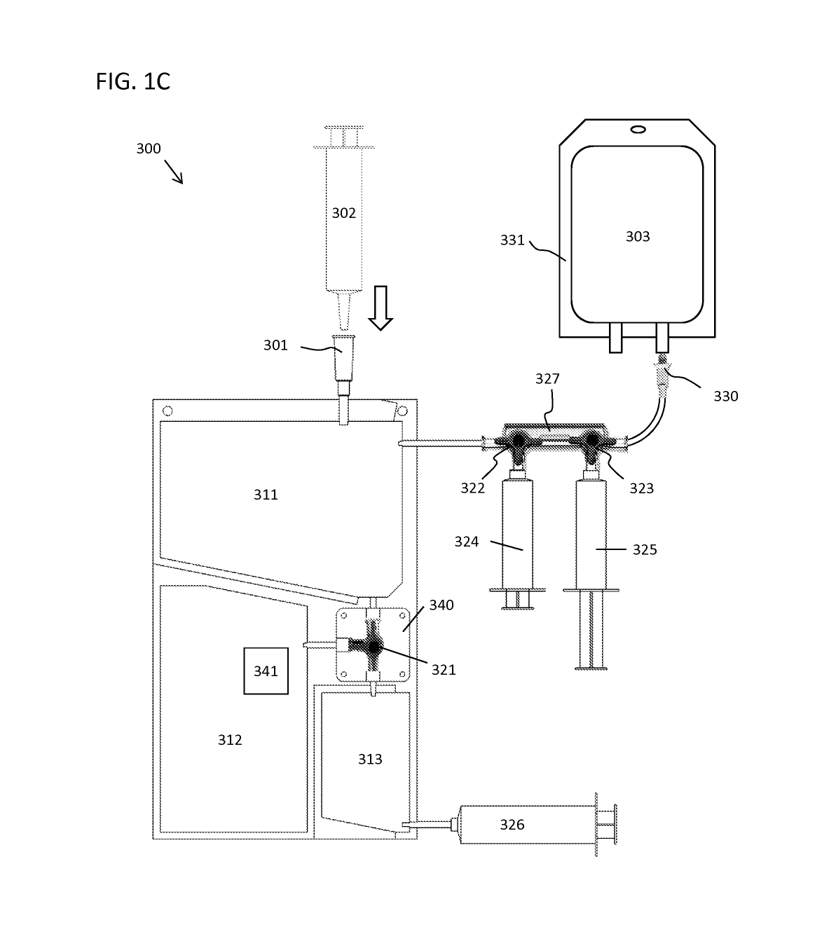

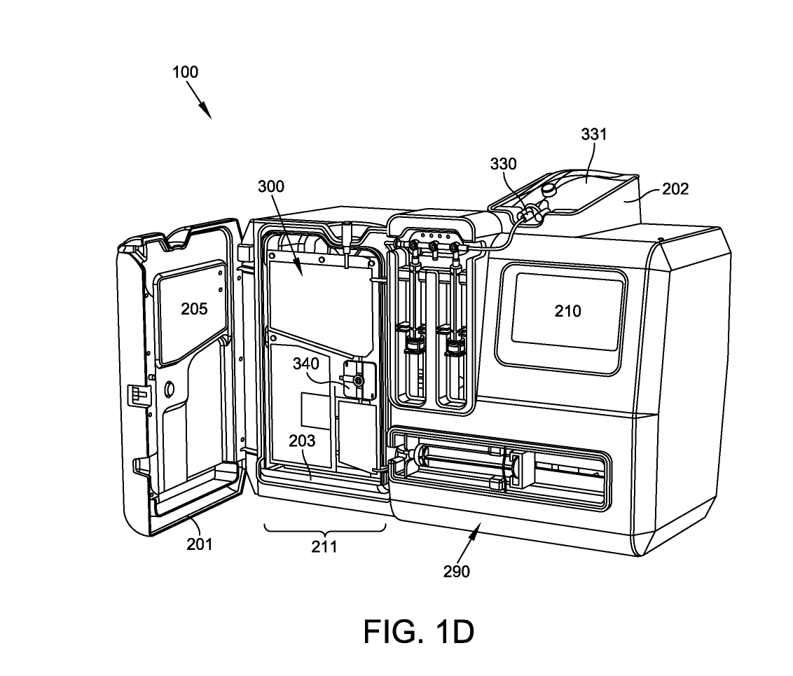

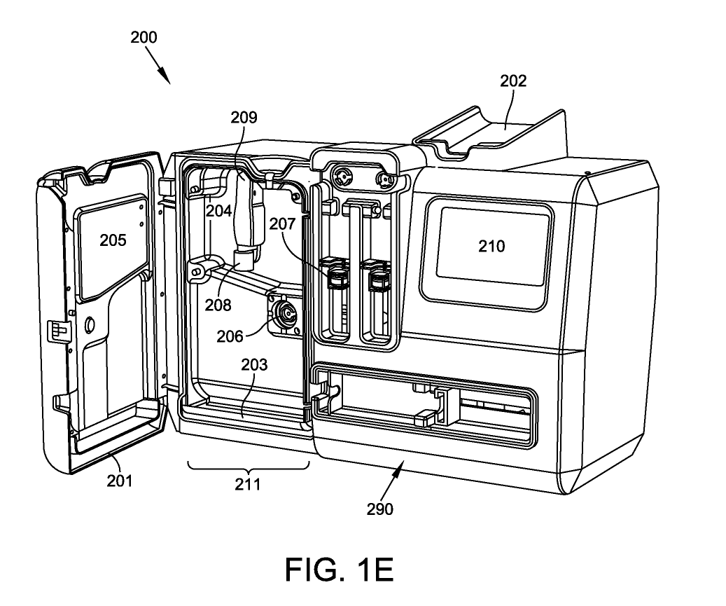

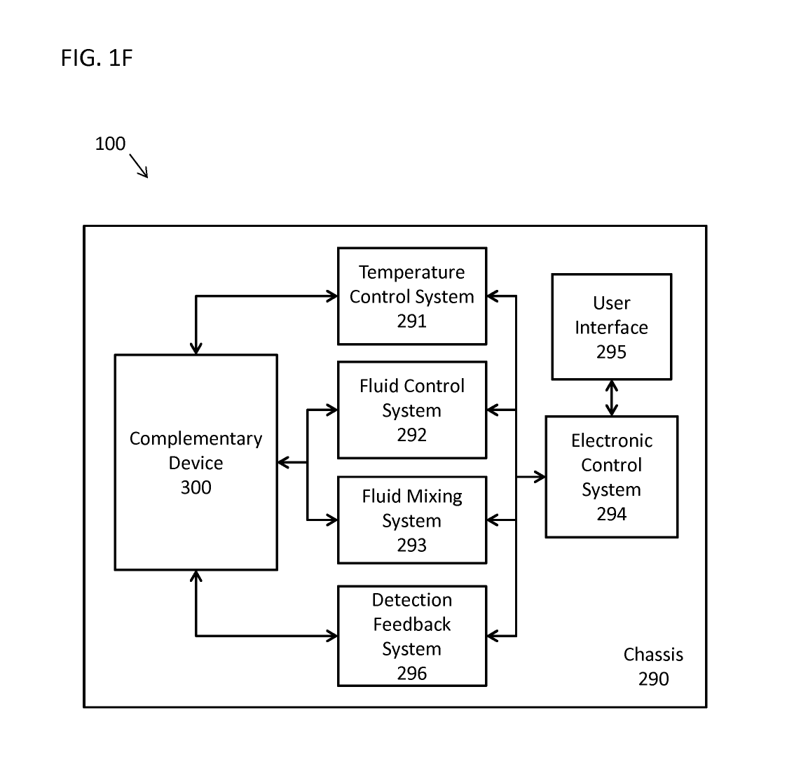

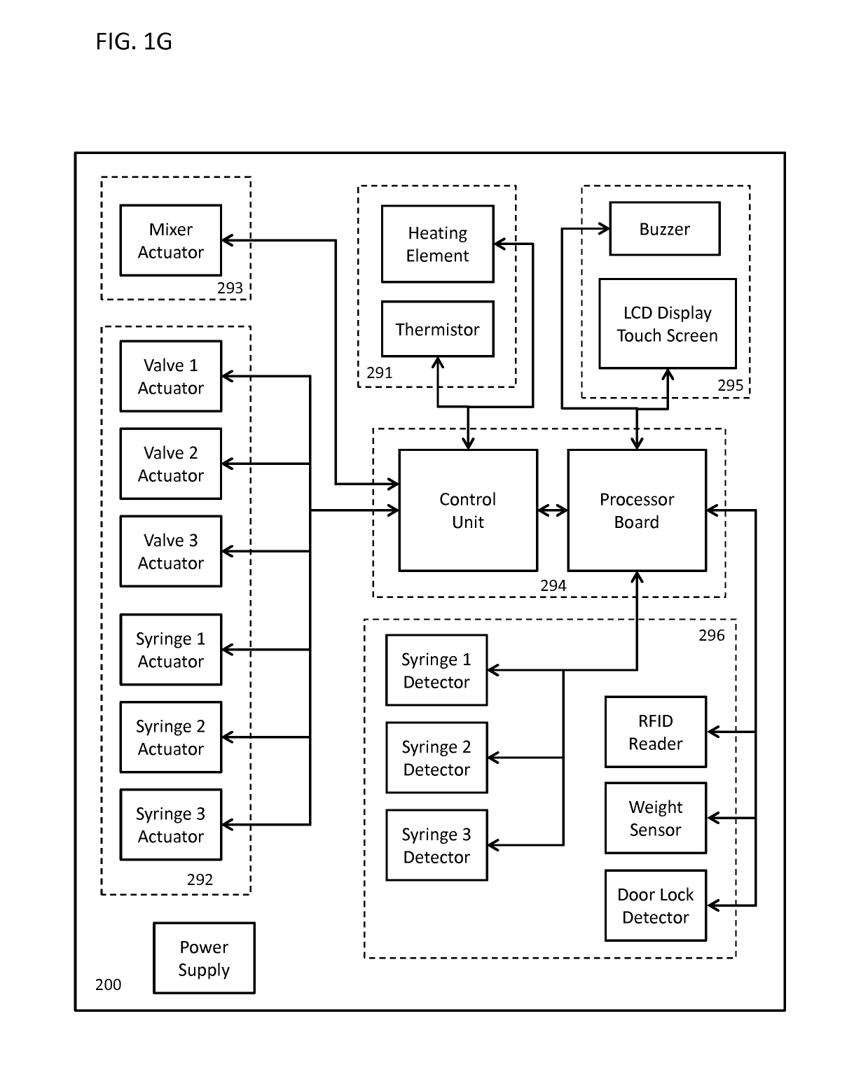

One embodiment of the present disclosure includes a system, generally indicated at 100 for automated tissue manipulation. An example of such a system is shown in FIGS. 1A-1G. The system 100 may be used to wash a tissue sample, extract cells from a tissue sample, treat a tissue sample, and/or prepare a sample for cryopreservation. The system 100 may also be programmed to perform other procedures known to persons skilled in the art. The system 100 includes a tissue manipulation machine or system 200 (FIG. 1B) and a complementary device 300 (FIG. 1C). The exemplary complementary device, shown schematically in FIG. 1C, includes an embodiment disclosed in International Publication WO 2013/086183 A1, which is herein incorporated by reference in its entirety for all purposes. The complementary device 300 may be sterile, single use, and configured to form a closed or semi-closed system, where a tissue sample can be manipulated with minimum risk of contamination. The complementary device 300 may further be individually packaged in a manner that facilitates its use in a clinical laboratory or an operating room.

The complementary device 300 may include a sample processing compartment 311 and a waste chamber 312 (FIG. 1C). The sample processing compartment 311 may be formed from a flexible material, for example, one or more sheets of a flexible plastic material. The sample processing compartment 311, waste chamber 312, and cell collection chamber 313, described below, as well as connecting conduits between these chambers may be disposed or formed between common sheets of flexible plastic material. The complementary device 300 may include an ID tag 341. To use the complementary device 300 for tissue washing, a tissue sample contained in a syringe 302 may be loaded to the sample processing compartment 311 via a sample inlet port 301. The syringe 302 may include a large tip opening, for example, a catheter tip or a Toomey tip, when the tissue sample contains large particles or agglomerations. The sample processing compartment 311 may contain a first filter mesh configured to retain the tissue sample. For example, a filter mesh having a pore size of from about 50 μm to about 400 μm may be used to retain a lipoaspirate sample. More specifically, the filter mesh may have pore sizes of from about 70 μm to about 200 μm, for example, about 70 μm, about 85 μm, about 100 μm, about 120 μm, about 140 μm, about 170 μm, or about 200 μm. A filter mesh having a pore size of about 10 μm, about 15 μm, about 20 μm, about 25 μm, about 30 μm, about 35 μm, about 40 μm, about 50 μm, about 60 μm, about 70 μm, about 85 μm, about 100 μm, about 125 μm, about 150 μm, about 175 μm, about 200 μm, about 250 μm, about 300 μm, about 400 μm, about 500 μm, about 600 μm, about 700 μm, about 800 μm, or about 1000 μm may also be used. While tissue pieces are retained in the sample processing compartment 311, excess fluids in the tissue sample, for example, blood, tumescent solution, and/or free oil from a lipoaspirate sample, may be drained into the waste chamber 312 through a first stopcock valve 321. The first stopcock valve 321 may be attached to a stopcock plate 340 which holds the stopcock 321 in position. The stopcock plate 340 may include holes, pins, and/or other structures to allow it to fit the stopcock 321 onto a tissue manipulation machine, for example, an embodiment of tissue manipulation machine 200, so that the stopcock 321 can be actuated precisely by the tissue manipulation machine.

In other embodiments, for example, embodiments configured to work with complementary devices having chambers that are fluidly connected by tubing or flexible conduits without an intervening stopcock valve, stopcock 321 may be augmented by or replaced by a mechanical actuator, for example, a pincer or a pincer-like structure, that may be actuated to pinch the tubing or flexible conduits closed. The tubing or flexible conduits of the complementary device may, in such embodiments, be considered valves of the complementary device.

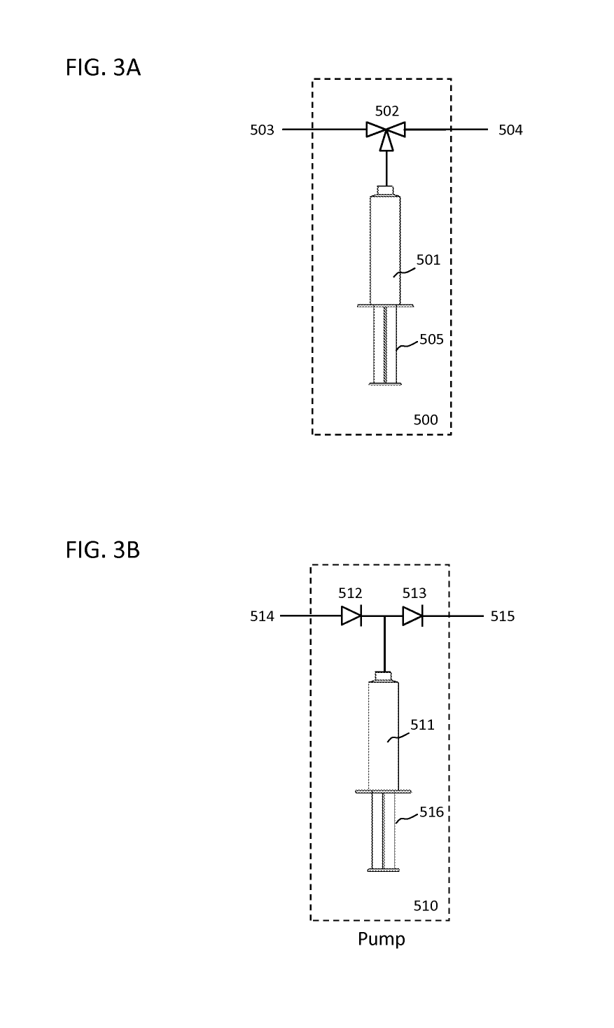

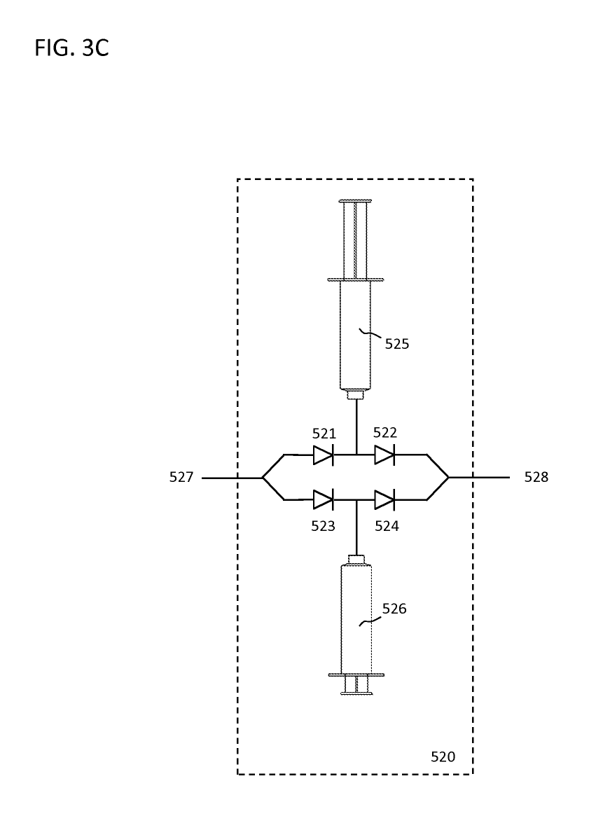

The tissue sample may be washed using a first solution 303, for example, a buffer solution, a saline solution, a rinse solution, a phosphate-buffered saline (PBS) solution, a culture medium solution, a Lactated Ringer's Injection solution (LRS), etc., which is fluidicly connected to the sample processing compartment 311 via a spike connector 330 and a stopcock manifold 327 comprising a second stopcock 322 and a third stopcock 323. The first solution 303 may be a rinse solution or a rinsing solution (used interchangeably herein); however, the functions of the first solution 303 may not be limited to rinsing. Precise volumes of the first solution 303 can be pumped into the sample processing compartment 311 using a syringe 324 attached to one of the stopcocks on the stopcock manifold 327, for example, the second stopcock 322. Alternatively, a syringe pump as illustrated in FIG. 3B may be used for pumping precise volumes of the first solution 303 into the sample processing compartment 311. Mixing actions may be provided to thoroughly wash the tissue sample by rocking, massaging, inverting and/or squeezing the sample processing compartment 311. A waste solution from the wash step may include fluids which are drained into the waste chamber 312. In another embodiment of the complementary device 300, the sample processing chamber 311 may contain no mesh filter.

The complementary device 300 may further be used for tissue dissociation, during which cells are released and extracted from a tissue sample. Tissue dissociation may be performed after one or multiple washes of a tissue sample in the sample processing compartment 311. Washing may increase dissociation efficiency and/or the purity of extracted cells. The system 100 may be configured to perform no wash, 1 wash step, 2 wash steps, 3 wash steps, 4 wash steps, 5 wash steps, 6 wash steps, or variable number of wash steps until a pre-determined level of sample cleanliness is reached. To perform tissue dissociation, a reagent solution, for example, a dissociation solution and/or a solution containing one or more enzymes, for example, collagenase, may be loaded in a syringe 325 attached to the stopcock manifold 327. The reagent solution may then be introduced into the sample processing compartment 311 through a stopcock 323. The sample processing compartment 311 may be agitated to facilitate mixing using rocking and/or massaging actions, and may be heated or cooled to, for example, about 37° C. for optimum tissue dissociation. After incubation for a period of time, for example, between about 3 minutes and about 240 minutes, about 3 min, about 5 min, about 10 min, about 15 min, about 20 min, about 25 min, about 30 min, about 35 min, about 40 min, about 45 min, about 50 min, about 55 min, about 60 min, about 75 min, about 90 min, about 105 min, about 120 min, about 150 min, about 180 min, or overnight, cells released from the tissue sample may be harvested in a syringe 326.

Optionally, a cell collection chamber 313 may be included in the complementary device 300 to collect the released cells. The collection chamber 313 may contain a second filter mesh, having a pore size of, for example, between about 10 μm and about 150 μm, about 10 μm, about 15 μm, about 20 μm, about 25 μm, about 30 μm, about 35 μm, about 40 μm, about 50 μm about 60 μm, about 70 μm, about 85 μm, about 100 μm, about 125 μm, or about 150 μm to reduce the amount of debris and clumps in the collected released cells. The refined cell population, i.e. the cells passing through the second filter mesh, may be optionally collected in a syringe 326 fluidicly coupled to the cell collection chamber 313. For some examples where cell clusters are to be isolated, such as hair follicles and pancreatic islets, larger pore size may be used, for example, between about 100 μm and about 600 μm, about 100 μm, about 150 μm, about 200 μm, about 250 μm, about 300 μm, about 400 μm, about 500 μm, or about 600 μm.

The complementary device 300 may further be used to treat a tissue sample. For example, a tissue sample may be treated with a cryoprotectant, for example, glycerol or dimethyl sulfoxide (DMSO), to prepare for cryopreservation. A portion of any excess fluids (for example, blood, buffer solution, tumescent solution, etc.) from the sample may be collected in the cell collection chamber 313 (also referred to herein as specimen chamber 313) as a specimen for bacterial counts and/or sterility culture tests for quality control purposes, for example, to determine whether the sample has been contaminated. The specimen chamber 313 may contain a filter mesh for removing debris, clumps, and/or factors that may interfere with testing. The tissue sample may then be washed using a rinsing solution 303 pumped into the sample processing compartment 311 using the syringe 325. The washed sample may further be treated with a cryoprotectant, which is preloaded in another syringe 324, under controlled temperature and/or gentle agitation. The cryoprotectant may be injected into the sample processing compartment 311 at a controlled rate. The sample processing compartment 311 may be cooled or temperature controlled during the process of injecting a cryoprotectant into the sample processing compartment 311 because some cryoprotectant such as DMSO may release heat upon mixing with the sample. Mixing actions, for example, massaging and/or rocking, may also or alternatively be performed on the sample processing compartment 311 while adding the cryoprotectant. It is appreciated that this process for tissue banking may be applied to many tissue types, for example, to lipoaspirate samples.

The various uses of the complementary device 300 disclosed herein may be combined, the orders of the processes disclosed herein may be altered, and the processes may be automated using a tissue manipulation machine disclosed herein. For example, tissue washing, dissociation (for example, enzymatic digestion), followed by debris and/or clump removal may be performed sequentially in one complementary device, which may provide a sterile closed-system environment and other significant advantages particularly when the processes are automated using a tissue manipulation machine. The stopcock manifold 327 may include additional stopcocks to accommodate as many syringes as desired. Additional reagents may be preloaded in those syringes. For example, the complementary device 300 may further comprise a 3-gang stopcock manifold having three stopcocks connected to a first syringe, a second syringe, and a third syringe. The first syringe may be used as a syringe pump to introduce an accurate volume of a first solution into the sample processing compartment 311, the second syringe may be preloaded with a first reagent, for example, a dissociation solution, an enzyme solution, or a collagenase solution, and the third syringe may be preloaded with a second reagent, for example, serum, autologous serum, plasma, or platelet rich plasma. This configuration may be used to wash a lipoaspirate sample, digest the lipoaspirate sample using a preloaded enzyme (for example, collagenase), and deactivate, neutralize, and/or quench the enzyme after digestion using a preloaded serum. The third syringe may be preloaded with a second dissociation solution, containing, for example, a second enzyme. This configuration may also be used to dissociate different parts of the tissue in steps, and release different cell types to be collected concurrently or at different times. The released cells may be passed through a cell strainer (for example, a filter mesh in the collection chamber 313) to remove clumps and debris. Multiple collection chambers may be configured to collect different cell types.

In another example, the complementary device disclosed in the present disclosure may be used to wash a tissue sample, dissociate the sample, and treat the released cells with a cryoprotectant in preparation for cryopreservation of the released cells. A tissue sample, for example, lipoaspirate, may be washed, digested with enzyme, optionally treated with a reagent (for example, serum) that inactivates (neutralizes and/or quenches) the enzyme, and/or mixed with a cryoprotectant in preparation for cryopreservation of the released cells (for example, SVF) in the complementary device.

One embodiment of the present disclosure includes a tissue manipulation machine that automates the processes that may be performed using a complementary device. Another embodiment of the present disclosure is a tissue manipulation machine that automates the processes that may be performed using embodiments of the complementary device 300 disclosed herein. A block diagram of an exemplary tissue manipulation machine of the present disclosure is shown in FIGS. 1F and 1G. The tissue manipulation machine 200 may comprise a mechanical frame 290, also referred to interchangeably herein as the chassis 290, a temperature control system 291, a fluid control system 292, a fluid mixing system 293, an electronic control system 294, a user interface 295, and optionally a detection feedback system 296 (FIG. 1F). The tissue manipulation machine 200 may provide accurate and precise process controls that lead to better quality, standardization, reproducibility, labor saving, sterility, and safety than prior known systems and apparatus. The tissue manipulation machine 200 may further provide automation to enable performance of complicated protocols or processes that are difficult to perform manually.

The chassis 290 provides physical structures to support the multiple control systems and at least one complementary device 300. The tissue manipulation machine 200 includes a chamber 211 configured to receive and retain a complementary device 300. In use, the chamber 211 may be sealed closed, for example, hermetically sealed, by door 201 of the tissue manipulation machine 200. In other embodiments, the chamber 211 may be at least partially open during use and at least a portion of the internal volume of the chamber 211 and/or at least a portion of a surface of a complementary device 300 disposed in the chamber 211 may be in communication with the atmosphere external to the machine 200. The tissue manipulation machine 200 includes a heating chamber 204 (FIG. 1E), which is part of the temperature control system 291, enclosed in a door 201. The heating chamber 204 and/or a temperature control sub-system 291 may be disposed within the chamber 211. During operation a complementary device 300 is mounted in the chamber 211 of the tissue manipulation machine 200 such that the sample processing compartment 311 is positioned in contact with, in, or proximate the heating chamber 204. In some embodiments, the complementary device 300 is loaded on the front side and/or the top of the tissue manipulation machine 200. The rinse solution bag 331 may be mounted on a tray 202, a plate and/or a surface on the tissue manipulation machine. The tray 202 may be tilted to allow the spike connector 330 to draw fluids from the bag 331 under the influence of gravity and/or by a pump. Alternatively, the rinsing solution bag 331 may be hung in a vertical position on a hook, or a structure comprising a pole and a hook. If heating or cooling of the rinsing solution is desired, the tray 202 and/or a surface in contact with the rinsing solution or rinse solution bag 331 may be configured to include a hot plate or cooling plate to provide temperature control. A cover over the tray 202 may also be provided to improve temperature uniformity of the rinse solution. In another embodiment, a semi-enclosed temperature controlled chamber may be included to house the rinse solution and/or rinse solution bag 331. Sensors, for example, a weighing scale, may be incorporated in the tissue manipulation machine 200 to detect whether the rinsing solution bag 331 is correctly mounted and/or whether the rinsing solution bag 331 has the correct weight and/or to provide an indication of an amount of rinsing solution 303 present in the rinsing solution bag 331. A weighing scale or a weight detector may also be used to detect the amount of rinse solution 303 added to the sample processing compartment 311. More specifically, amounts of rinse solution added to the sample processing compartment 311 may be accurately controlled using a valve and gravity feed. For example when 30 g of a saline solution needs to be added to the sample processing compartment 311, the valve may open to let the saline solution flow under gravity until the saline solution bag becomes 30 g lighter.

In another embodiment, the tissue manipulation machine 200 includes a surface to place at least one complementary device 300. The surface may serve as a hot plate or cool plate to control the temperature of at least a portion of the complementary device.

The chassis 290 may also provide mechanical structures to support components including but are not limited to actuators, sensors, heater elements, electronic circuit boards, a built-in computer, power supplies, and/or a touch screen. For certain applications, for example, clinical applications, the chassis 290, or at least a portion of the chassis 290 may be configured to be water resistant and/or disinfectant resistant, using materials compatible with common disinfectants, for example, 70% ethyl alcohol and 10% bleach. The exterior panels of the chassis 290 may be fabricated as one piece to reduce the number of seams and/or openings that may be exposed to accidental spills during use. The chassis 290 may be configured so that certain critical surfaces can be easily wiped down or even sprayed down with a disinfectant. The chassis 290 may be configured to serve as secondary containment in case of accidents where the complementary device 300 (primary containment) is breached or compromised. This feature may be particularly useful in a clinical setting, where tissue sample leakage or spillage may be a potential biohazard.

The exemplary tissue manipulation machine 200 may enclose the sample processing compartment 311 in the heating chamber 204. The tissue manipulation machine 200 may further include a removable tray 203 configured to collect any potential leak from the complementary device 300 in case of accidents where the complementary device 300 is breached or compromised. The tray 203 may be pulled out, cleaned and disinfected easily.

The temperature control system 291 may comprise a temperature control chamber 204, also referred to herein as a heating chamber 204, a heater element, at least one temperature detector, and optionally, a cooling element. In some embodiments, a cooling element may be used to cool a portion of a complimentary device 300, for example, the sample processing compartment 311. Embodiments of the temperature control chamber 204 may not be limited to heating only. In the exemplary tissue manipulation machine 200, the temperature control system 291 may be configured to maintain the temperature of the temperature control chamber 204 and the sample processing compartment 311 at one or more predetermined values, for example, between about 4° C. and about 60° C., between about 18° C. and about 45° C., between about 25° C. and about 42° C., between about 34° C. and about 38° C., between about 35° C. and about 37° C., between about 36° C. and about 37.5° C., at about 37° C., or around a temperature where the dissociation has the highest efficacy. Specifically, the temperature control system 291 may be configured to provide temperatures that are optimum for the process to be performed. For example, for enzymatic digestion, the temperature control chamber 204 may be heated to between about 34° C. and about 38° C. More specifically, the temperature control chamber 204 may be heated to between about 35° C. and about 37° C., or about 37° C. for enzymatic digestion using collagenase. In another example, for adding DMSO to a sample for preparation for cryopreservation, the temperature control chamber 204 may be cooled to between about 0° C. and about 12° C., between about 2° C. and about 8° C., around 4° C., or below about 18° C. In some embodiments, the temperature control chamber 204 may be cooled to about 4 degrees Celsius during mixing a sample with DMSO. At least one portion of the temperature control chamber 204 may be thermally insulated to enhance temperature uniformity. A temperature within the temperature control chamber 204, particularly within the sample processing compartment 311, may be maintained with a variation of less than about 6° C., less than about 4° C., less than about 3° C., less than about 2° C., less than about 1 degree Celsius, less than about 0.5° C., or less than about 0.2° C. During heating or cooling, temperature control chamber 204 may achieve a rapid temperature change rate of, for example, greater than about 1 degree Celsius per minute, greater than about 2° C. per minute, greater than about 3° C. per minute, greater than about 4° C. per minute, greater than about 5° C. per minute, greater than about 6° C. per minute, greater than about 7° C. per minute, greater than about 8° C. per minute, greater than about 10° C. per minute, greater than about 12° C. per minute, greater than about 15° C. per minute, greater than about 18° C. per minute, greater than about 20° C. per minute, greater than about 25° C. per minute, greater than about 30° C. per minute, greater than about 40° C. per minute, greater than about 50° C. per minute, greater than about 60° C. per minute, greater than about 80° C. per minute, or greater than about 100° C. per minute.

In the exemplary tissue manipulation machine 200, the heater element may comprise a hot plate 205 comprising a heating element, for example, an etched pad heating element, a nichrome wire, ribbon or strip, a resistance wire or coil, an etched foil, a radiative heating element (for example, a heat lamp), a peltier element, a peltier plate, etc. The hot plate 205 may further comprise a thermal mass that has good thermal conductivity, for example an aluminum plate, a copper plate, and/or a circulating fluid mass, to diffuse the heat generated from the heating element for good temperature uniformity across the hot plate 205. More than one heater element may be used to create uniform temperature profiles in the temperature control chamber 204. The hot plate 205 may preferably be configured to be in direct contact with the complementary device 300, for example, the sample processing compartment 311, to ensure good thermal transfer. The temperature control system 291 may comprise at least one cooling element, for example, a refrigerator compressor, a peltier element, a peltier plate, and/or a thermo-electric device, configured to generate a temperature lower than the ambient operating temperature. The cooling element may be incorporated into the hot plate 205. A heat sink may be used to dissipate heat removed by the cooling element.

Another configuration of a temperature control system 291 may comprise a forced air system, having a temperature control chamber 204 optionally including an air vent, an air intake, and an air duct system that allows forced air to circulate substantially within the system. A fan may be used to drive the air circulation. A heating element, optionally a cooling element, and optionally a filter that removes dust particles from the circulating air, may also be included in the temperature control system 291. The forced air pathways, including the temperature control chamber 204 and the optional air ducts, may be thermally insulated. Thermal insulation may be achieved using thermal insulation materials such as thermal insulation foam, or a vacuum chamber. A forced air temperature control system may be configured to provide a uniform temperature profile within the temperature control chamber 204.

It may not be necessary to enclose the complimentary device in a chamber to heat or cool the complimentary device. In one embodiment a portion of the complimentary device is in direct contact with a heating plate without being enclosed in a heating chamber. In another embodiment a complimentary device is mounted on a tissue manipulation machine without being enclosed in a chamber. The tissue manipulation machine may generate a temperature controlled forced air flow that blows over a portion of the complimentary device to control the temperature of fluids in the portion of the complimentary device. In one embodiment of the present disclosure, a tissue manipulation system comprising a tissue manipulation machine and a complimentary device achieves rapid and uniform heating of contents in a sample processing chamber of the complimentary device using a high surface to volume ratio design of the sample processing chamber and the temperature control system. For example, a sample processing chamber of the complimentary device may comprise of a pouch made of flexible plastic sheets such as polyvinyl chloride (PVC) and polyurethane (PU), having a width of 15 cm and a height of 10 cm may accommodate about 60 ml of contents comprising a tissue sample and fluids. The inner surface of the processing chamber is about 300 cm², and the surface to volume ratio is about 5 cm⁻¹. In contrast, a standard centrifuge test tube having an inner volume of about 50 ml has a height of about 10 cm and an inner diameter of about 2.5 cm. The surface area of the test tube is about 88 cm² and the surface to volume ratio is about 1.76 cm⁻¹. The sample processing chamber in this example may be much easier to heat up in a rapid and uniform fashion compared to the standard centrifuge test tube as the surface to volume ratio of the complimentary device is about 3 times larger than that of the test tube. A large surface to volume ratio may ensure that for every volume of content to be heated, a large amount of surface of the sample processing chamber may be used to transfer heat, resulting in rapid and uniform heating. Further, in a large surface to volume ratio design, the fluid and tissue sample contents may be spread thinner than in a low large surface to volume ratio configuration, further reducing the time needed for heat to transfer throughout the contents, increasing the heating speed and uniformity. A high surface to volume ratio sample processing compartment configuration allows for rapid and uniform cooling as well. Mixing may be applied to the sample processing chamber to further increase the speed of heat transfer through convection of fluids in the chamber, resulting in rapid heating and uniform temperature distributions in the sample processing chamber. The processing chamber of the complimentary device may have an inner surface to volume ratio of about 1.5 cm⁻¹, about 2 cm⁻¹, about 2.5 cm⁻¹, about 3 cm⁻¹, about 4 cm⁻¹, about 5 cm⁻¹, about 6 cm⁻¹, about 8 cm⁻¹, about 10 cm⁻¹, about 15 cm⁻¹, about 20 cm⁻¹ or larger. The combination of efficient exposure to a heating or cooling source and good mixing may facilitate rapid and uniform heating and cooling. However, in many configurations, there may be significant trade-offs, constrains and limitations between efficient contact with the temperature source, efficient mixing, and/or other factors. For example, in a solid container (for example a test tube or a syringe), contents get heated when in contact with the inner surface of the container. Maximizing contact with the heat source may mean filling up the container rather full. However, filling the container reduces the air and free space in the container facilitate efficient mixing in a rocking or inverting mixing configuration, resulting in sluggish temperature control and non-uniform temperature distribution in the container. The exemplary tissue processing system disclosed herein (shown in FIG. 1 for example) may overcome such limitations by employing a high surface to volume ratio and flexible pouch as the sample processing compartment. The high surface to volume ratio allows for large contact area to the heating/cooling source resulting in rapid response to temperature control, whereas the flexibility allows for using massaging action to enable efficient mixing, thereby simultaneously achieving rapid and uniform heating or cooling.

In one embodiment of the present disclosure, a tissue manipulation system comprising a tissue manipulation machine and a complimentary device may heat and/or cool the fluid and tissue sample content in a compartment of the complimentary device rapidly and uniformly, and maintain the temperature within a tight range for a period of time, wherein the content in the compartment is at least 10 ml, 15 ml, 20 ml, 25 ml, 30 ml, 35 ml, 40 ml, 50 ml, 60 ml, 70 ml, 75 ml, 80 ml, 90 ml, 100 ml, 110 ml, 120 ml, 130 ml, 150 ml, 175 ml, or at least 200 ml, wherein the heating rate is at least 1° C./min, 1.2° C./min, 1.5° C./min, 2° C./min, 2.5° C./min, 3° C./min, 4° C./min, 5° C./min, 6° C./min, 7° C./min, 8° C./min, 9° C./min, 10° C./min, 12° C./min, 15° C./min, 20° C./min, 25° C./min, 30° C./min, 40° C./min, or 50° C./min, and wherein the temperature is maintained within 3° C., 2.5° C., 2° C., 1.5° C., 1.2° C., 1° C., 0.8° C., 0.7° C., 0.6° C., 0.5° C., 0.4° C., 0.3° C., 0.2° C. or 0.1° C., for a period of at least 1 minute, 2 minutes, 3 minutes, 5 minutes, 7 minutes, 10 minutes, 12 minutes, 15 minutes, 20 minutes, 25 minutes, 30 minutes, 35 minutes, 40 minutes, 45 minutes, 50 minutes, 60 minutes, 75 minutes, 90 minutes, 105 minutes, 120 minutes, 150 minutes, or 180 minutes. In another embodiment of the present disclosure, a tissue manipulation system comprising a tissue manipulation machine and a complimentary device is capable of heating at least 10 ml, 15 ml, 20 ml, 25 ml, 30 ml, 35 ml, 40 ml, 50 ml, 60 ml, 70 ml, 75 ml, 80 ml, 90 ml, 100 ml, 110 ml, 120 ml, 130 ml, 150 ml, 175 ml, or at least 200 ml of content in a sample processing chamber of the complimentary device to a target temperature of between about 34° C. and about 39° C., for example, about 34° C., about 35° C., about 36° C., about 37° C., about 38° C., or about 39° C., within a short period of time, for example, less than about 5 minutes, about 5 minutes, about 4 minutes, about 3 minutes, about 2 minutes, about 1 minute, about 50 seconds, about 40 seconds, about 30 seconds, about 25 seconds, about 20 seconds, about 15 seconds, about 10 seconds, or about 5 seconds, and maintain the temperature within a tight range of between about 0.1° C. and about 3° C., for example, about 3° C., 2.5° C., 2° C., 1.5° C., 1.2° C., 1° C., 0.8° C., 0.7° C., 0.6° C., 0.5° C., 0.4° C., 0.3° C., 0.2° C. or about 0.1° C. for a period of time of between about 1 minute and about 180 minutes, for example, about 1 minute, about 2 minutes, about 3 minutes, about 5 minutes, about 7 minutes, about 10 minutes, about 12 minutes, about 15 minutes, about 20 minutes, about 25 minutes, about 30 minutes, about 35 minutes, about 40 minutes, about 45 minutes, about 50 minutes, about 60 minutes, about 75 minutes, about 90 minutes, about 120 minutes, about 150 minutes, or about 180 minutes. In yet another embodiment of the present disclosure, a tissue manipulation system comprising a tissue manipulation machine and a complimentary device may control the temperature variation of the content in a sample processing chamber of the complimentary device to be within 2° C., 1.5° C., 1.2° C., 1° C., 0.8° C., 0.7° C., 0.6° C., 0.5° C., 0.4° C., 0.3° C., 0.2° C., 0.15° C. or 0.1° C. when a target temperature range is reached. Because the dissociation reagent solution may be temperature sensitive, for example, the enzyme activity of collagenase may be maximum at about 37° C., the ability to control temperature rapidly and uniformly may advantageously result in more efficient tissue dissociation and processing, high cell viability, high cell recovery, and short processing time.

The temperature control system 291 may further include components for heating or cooling the rinsing solution 331 and/or the reagents contained in a reagent syringe, for example, the syringe 325. In one embodiment, the rinsing solution tray 202 comprises a hot plate configured to warm and/or cool the rinsing solution, to, for example, between about 25° C. and about 45° C., between about 32° C. and about 40° C., about 32° C., about 35° C., about 36° C., about 37° C., or about 40° C. In another embodiment, the temperature control system 291 is configured to heat a dissociation solution, for example, a dissociation solution loaded in a syringe 325, to between about 30° C. and about 40° C., about 30° C., about 32° C., about 34° C., about 36° C., or about 37° C., using, for example, forced air or any heating methods known in the art.

At least one temperature detector, for example, a thermistor, a thermometer, a thermocouple or another type of temperature detector known in the art, may be positioned at or around the temperature control chamber 204 to measure the temperature in the temperature control chamber 204. A high precision thermistor may be utilized when accurate temperature measurements are required. The temperature information may be provided to a controller, which may be included in the electronic control system 294, which controls at least one control element (for example, a heating element, a cooling element, and/or a fan) using a control loop algorithm, which calculates an error value as the difference between a measured temperature and a pre-programmed set point. The controller may minimize the error by adjusting the power provided to the control element. The controller may be a proportional-integral-derivative controller (PID controller), where the proportional, the integral and the derivative of the error values, denoted P, I, and D, are calculated based on the current rate of temperature change to estimate the present error, the accumulation of past errors, and a prediction of future errors, respectively. A weighted sum of these three errors, or generally a mathematical combination of these three errors, may then be used to adjust the power sent to the at least one control element. Other controllers known in the art may also be used. The power sent to the control element may be modulated using amplitude modulation, pulse modulation, pulse width modulation, pulse amplitude modulation, or any other modulation method known in the art. The controller may be configured to minimize or avoid temperature overshoots. The temperature control system 291 may be programmed to perform a certain temperature profile, where the temperatures are set to different values at different time points.

In one embodiment of the present disclosure a tissue manipulation system comprises an electronic control system, a temperature control system and a fluid mixing system configured for rapid, uniform and accurate temperature control (heating and/or cooling).

The fluid control system 292 may include actuated valves and pumps comprising linear actuators and/or rotation actuators. A valve included in the fluid control system 292 may comprise a stopcock on a complementary device 300 actuated by a rotation actuator on a tissue manipulation machine 200. For example, the stopcock 321 on the complementary device 300 may be actuated by a rotation actuator 206 on a tissue manipulation machine 200. A pump included in the fluid control system 292 may comprise a syringe on a complementary device 300 and a linear actuator on a tissue manipulation machine 200, for example, the syringe 324 and the linear actuator 207. The fluid control system 292 may include a plurality, for example, two or more linear actuators 207 to individually pump fluid from a plurality of syringes, for example, syringes 324 and 325 of an embodiment of the complimentary device 300.

In another embodiment, the fluid control system 292 may comprise other configurations, for example, including one or more passive components on a complementary device 300 driven by an active component, for example, an actuator, on a tissue manipulation machine 200. The advantages of such embodiments include that the component in direct contact with fluids are part of the complementary device 300, which may be sterile, single use, and a closed system. In yet another embodiment, the fluid control system 292 may include a pinch valve, a peristaltic pump, a check valve, a duckbill valve, a syringe pump, a positive displacement pump, a reciprocating pump, a rotary pump, and/or other fluid control elements known in the art. In one embodiment, at least one sensor, for example, an optical sensor, an electrical capacitance sensor, an ultrasound detector, a flow meter, a pressure sensor, or a Doppler flow detector may be used to detect the flow rates of fluids, the properties (for example, color, turbidity, light absorption, viscosity, etc.) of fluids including the tissue sample, and clogging of the fluid lines, etc. The detected information may be provided to the electronic control system 294 used to control the tissue manipulation machine 200 and/or trigger a pre-programmed response.

In another embodiment, the fluid control may use gravity. For example, fluids may be injected or drained through a valve, such as a stopcock, a check valve or a pinch valve. The amount of fluids transferred may be controlled by the time the valve is open or closed. The amount of fluids transferred may also be controlled by measuring the weight of a chamber. For example, the weight of a solution contained in a chamber, a container, or a bag may be measured before the valve is open. The valve is then opened to allow fluid flow until the weight of the solution becomes a pre-determined amount less.

A rotation actuator included in the fluid control system 292 may comprise a stepper motor. In some embodiments, a stepper motor may be controlled in open loop (no position feedback) with good accuracy using a large gear ratio, for example, a gear ration of between about 10:1 and about 500:1, about 10:1, about 15:1, about 20:1, about 25:1, about 30:1, about 40:1, about 50:1, about 60:1, about 80:1, about 100:1, about 120:1, about 150:1, about 200:1, about 250:1, about 300:1, about 400:1, or about 500:1. In another embodiment, a rotation actuator comprises a stepper motor in a closed loop (with position feedback) configuration. In yet another embodiment, a brushed DC motor coupled with a gearbox may be used as an actuator. A linear actuator may also comprise a stepper motor, in close loop or open loop configuration. An encoder may be used with an actuator to provide position information for accurate closed loop control. A limit switch, for example, an infrared limit switch or an optical limit switch, may be used to determine the position of an actuator. In yet another embodiment, pneumatic actuators are used in the fluid control system 292. Other actuators known in the art, such as various types of hydraulic actuators, pneumatic actuators, electric actuators and/or mechanical actuators may also be used. An end stop, for example, an infrared end stop or an optical end stop, may be used to identify absolute positions of a linear actuator.

The fluid mixing system 293 in embodiments of the exemplary tissue manipulation machine 200 may comprise a roller 208 on swinging arm 209, which swings back and forth driven by a rotational actuator. Alternatively, the roller 208 may be mounted on a linear actuator. The roller 208 may be configured to press a portion of the sample processing compartment 311 against or towards the hot plate 205, and/or to provide massaging actions to agitate and mix fluids inside the sample processing compartment 311. The roller speed may be optimized according to the sample and the process to be performed. For example, the roller may be controlled to move at a speed of between about 1 cm/sec and about 200 cm/sec, for example, at about 200 cm/sec, about 100 cm/sec, about 60 cm/sec, about 45 cm/sec, about 30 cm/sec, about 20 cm/sec, about 15 cm/sec, about 10 cm/sec, about 7 cm/sec, about 5 cm/sec, about 3 cm/sec, about 2 cm/sec, or about 1 cm/sec. The roller may be configured to move at a frequency of between 0.2 Hz and 3 Hz, for example about 0.2 Hz, about 0.3 Hz, about 0.4 Hz, about 0.5 Hz, about 0.6 Hz, about 0.7 Hz, about 0.8 Hz, about 0.9 Hz, about 1 Hz, about 1.1 Hz, about 1.2 Hz, about 1.3 Hz, about 1.5 Hz, about 1.7 Hz, about 2 Hz, about 2.2 Hz, about 2.5 Hz, and/or about 3 Hz. For processing of lipoaspirate, the roller may move at a linear speed of between about 3 cm/sec and about 30 cm/sec. Other mixing mechanisms known in the art may also be used. In an alternative embodiment, the fluid mixing system 293 comprises a rotating arm that presses against one surface of the sample processing compartment 311. In another embodiment, the fluid mixing system comprises two rotating arms that press against or into one surface of the sample processing compartment 311 and that rotate in, for example, opposite directions. In yet another embodiment, the fluid mixing system 293 comprises at least one moving plate that presses periodically on a portion of the sample processing compartment 311. In yet another embodiment, the fluid mixing system 293 comprises a shaker that shakes or rocks the sample processing compartment 311. In yet another embodiment, the fluid mixing system 293 comprises an ultrasonic transducer which applies ultrasonic energy to a sample in the sample processing compartment 311.

In yet another embodiment, the fluid mixing system 293 comprises a mechanism that periodically inverts the sample processing chamber 311. For fluid mixing systems that rely on massaging and/or deforming a complementary device, for example, the roller based, the rotating arm based, and the moving plate based systems disclosed herein, the mixing mechanism may be positioned at a distance from the heating plate. In one embodiment, a fluid mixing system comprises a roller 208 configured to press into a sample processing compartment pouch of a complementary device 300 against a heating plate 205 (FIG. 1D). The roller may be mounted on a spring loaded arm. The roller may apply a force and/or a pressure on the sample processing compartment. The roller may press against the heating plate. Alternatively, the roller may be positioned to leave a gap from the heating plate. The roller may be configured to keep a substantially constant distance from the heating plate, or may be configured to vary the distance from the heating plate depending on the position of the roller. The minimum distance between the roller and the heating plate may be between about 0 mm and about 40 mm, for example, about 0 mm, about 0.5 mm, about 1 mm, about 1.5 mm, about 2 mm, about 2.5 mm, about 3 mm, about 3.5 mm, about 4 mm, about 5 mm, about 6 mm, about 7 mm, about 8 mm, about 9 mm, about 10 mm, about 12 mm, about 15 mm, about 18 mm, about 20 mm, about 25 mm, about 30 mm, or about 40 mm to achieve high efficacy mixing. In one embodiment the distance between the roller and the heating plate may be between 1 mm and 6 mm. In another embodiment the distance between the roller and the heating plate may be between 2 mm and 5 mm. In yet another embodiment the distance between the roller and the heating plate may be between 3 mm and 10 mm. In yet another embodiment the distance between the roller and the heating plate is smaller than 1 mm. For effective dissociation of tissues that have significantly different density than the dissociation solution, for example, a fat tissue in an aqueous enzyme solution, mixing may facilitate efficient reaction and dissociation, as the tissue may otherwise separate from the dissociation solution due to different buoyancies. However, over mixing may damage tissue leading to low cell viability and recovery. The fluid mixing system may include an actuator, such as a roller, a moving arm, and/or a moving plate, which agitates at a frequency of between 0.1 Hz and 5 Hz, for example about 0.1 Hz, about 0.2 Hz, about 0.3 Hz, about 0.4 Hz, about 0.5 Hz, about 0.6 Hz, about 0.7 Hz, about 0.8 Hz, about 0.9 Hz, about 1 Hz, about 1.1 Hz, about 1.2 Hz, about 1.3 Hz, about 1.5 Hz, about 1.7 Hz, about 2 Hz, about 2.2 Hz, about 2.5 Hz, about 3 Hz, about 4 Hz, or about 5 Hz.

The fluid mixing system 293 may be programmed to execute specific mixing profiles. Agitation strength, amplitude, speed, and/or frequency may be varied as a function of time. For example, the fluid mixing system may perform intermittent agitation, speed varying agitation, etc. using electronic or computer control. These mixing profiles may be difficult to perform manually, especially with accuracy and reproducibility. One agitation profile useful for tissue dissociation may be to agitate rigorously at the beginning (first phase of mixing) of a dissociation step, and to agitate mildly (second phase of mixing) towards the end of the dissociation step. For example, using a roller, the first phase of mixing may be carried out at a speed of between about 20 cm/sec and about 80 cm/sec, about 20 cm/sec, about 30 cm/sec, about 50 cm/sec, or about 80 cm/sec, for between about 3 min and about 20 min, about 3 min, about 5 min, about 10 min, about 15 min, or about 20 min. The second phase of mixing may be carried out at a speed of between about 3 cm/sec and about 15 cm/sec, about 3 cm/sec, about 5 cm/sec, about 10 cm/sec, or about 15 cm/sec, for between about 10 min and about 60 min, about 10 min, about 15 min, about 20 min, about 30 min, about 45 min, or about 60 min. In another example, the first phase of mixing may be carried out continuously and the second phase intermittently.

Another agitation profile useful for tissue dissociation may comprise many cycles of agitation each comprising a first phase of one speed and/or frequency followed by a second phase of a different speed and/or frequency. For example, in the first phase, the agitation frequency may be between 0.3 Hz and 3 Hz, for example about 0.3 Hz, about 0.4 Hz, about 0.5 Hz, about 0.6 Hz, about 0.7 Hz, about 0.8 Hz, about 0.9 Hz, about 1 Hz, about 1.1 Hz, about 1.2 Hz, about 1.3 Hz, about 1.4 Hz, about 1.5 Hz, about 1.6 Hz, about 1.8 Hz, about 2 Hz, about 2.2 Hz, about 2.5 Hz, or about 3 Hz, whereas in the second phase, the agitation frequency may be between 0 Hz (no agitation) and 2 Hz, for example no agitation, about 0 Hz, about 0.05 Hz, about 0.1 Hz, about 0.15 Hz, about 0.2 Hz, about 0.3 Hz, about 0.4 Hz, about 0.5 Hz, about 0.6 Hz, about 0.7 Hz, about 0.8 Hz, about 0.9 Hz, about 1 Hz, about 1.1 Hz, about 1.2 Hz, about 1.3 Hz, about 1.4 Hz, about 1.5 Hz, about 1.6 Hz, about 1.8 Hz, or about 2 Hz. The duty cycle of the first phase may be between 1% and 80%, between 5% and 60%, between 5% and 20%, between 5% and 30%, or between 10% and 25%. For example, the duty cycle of the first phase may be about 3%, about 5%, about 8%, about 10%, about 12%, about 15%, about 20%, about 25%, about 30%, about 33%, about 40%, about 50%, about 60%, or about 75%. In another embodiment the mixing profile may comprise cycles including a strong agitation phase and a weak agitation phase, wherein the weak agitation phase may include no agitation. The strength of agitation may be controlled by at least one of the agitation speed, roller speed, agitation frequency, the amplitude of agitation, the distance of the mixing mechanism such as a roller from the heating plate, the amount of fluids in the sample processing chamber displaced, and the force on which the mixing mechanism exerts on the sample processing chamber. In yet another embodiment, the mixing profiles may comprise bursts of agitation using, for example, massaging, mixing, and/or rocking, at a random interval. In yet another embodiment, the mixing profiles may comprise mixing at variable speeds, frequencies, intensities and/or duty cycles. In yet another embodiment, the mixing profiles may comprise mixing at random speeds, frequencies, intensities, and/or duty cycles. In yet another embodiment, the mixing profiles may comprise mixing at periodic speeds, frequencies, intensities, and/or duty cycles.

In one embodiment, at least one sensor, for example, an optical sensor, an electrical capacitance sensor, and/or an ultrasound sensor, may be used to detect the extent of dissociation and provide feedback to the electronic control system 294 to adjust the strength, speed, and/or frequency of mixing actions accordingly.

The detection feedback system 296 comprises sensors configured to detect the status of the complementary device 300. The sensors may comprise those known in the art, including, but not limited to, mechanical or optical limit switches, infrared (IR) limit switches, weight sensors, temperature sensors, pressure sensors, fluid pressure sensors, flow sensors, etc. Embodiments of the detection feedback system 296 are designed to minimize error, and provide an interactive user experience. A weighing scale or a weight detector may be incorporated in the tissue manipulation machine 200 to detect whether a correct rinsing solution 303 is mounted. A pressure sensor may be used to detect fluid connection and clogging. An optical sensor may be used to detect whether a tissue sample is thoroughly washed by detecting the color and/or measuring the turbidity of the tissue sample. Electrical sensors may be used to detect changes in capacitance, for example, on two locations on the sample processing compartment 311 of a complementary device 300, to determine whether a tissue sample has been thoroughly dissociated. The detection feedback system 296 in the exemplary tissue manipulation machine 200 may comprise sensors to detect the status of the complementary device 300, including, for example, whether the complementary device 300 is properly mounted in the tissue manipulation machine 200, whether the syringes 324, 325, 326 are in position, whether the door 201 is closed, and optionally whether the rinsing solution 303 is in place on the tray 202. The tissue manipulation machine 200 may include a door lock that can be controlled by the electronic control system 294. The door 201 may be automatically locked during a run to prevent accidental interruption. The tissue manipulation machine 200 may further include a door lock detector to detect the status of the door (for example, open or closed or locked or unlocked). In one embodiment, shown in FIG. 1G, the detection feedback system 296 includes syringe detectors comprising, for example, limit switches (either mechanical or optical), a radio frequency identification (RFID) reader/writer, and optionally, a weight sensor. Because the presence of the syringes 324, 325 may be important for the successful performance of a process, the detection feedback system 296 may be configured to detect the presence of the syringes 324, 325 during the process. Information obtained by the detection feedback system 296 may be sent to the electronic control system 294 to monitor the status of the presence of the syringes 324, 325, and react according to a pre-determined (programmed) procedure when a syringe 324, 325 is detected to be absent. For example, a warning message may be displayed on a screen 210, an error message may be logged to a log file, a buzz may sound using a buzzer to notify a user, and/or a process may be aborted or paused until the situation is resolved. Other actions may be taken in a pre-programmed manner when the detection feedback system 296 detects an error.

In one embodiment, the complementary device 300 includes an identification (ID) tag 341, which may contain information, referred to herein as the ID information, such as a serial number, a set of process parameters, and/or information that determines the process or protocols to be executed by the tissue manipulation machine 300. The identification (ID) tag 341 may comprise a radio-frequency identification (RFID) tag, a barcode, a linear barcode, a matrix (2D) barcode, or any tissue manipulation machine-readable representation of ID or a memory device known in the art. The tissue manipulation machine 200, in particular, the detection feedback system 296 thereof, may include an ID reader, which reads the ID information of a complementary device 300 being loaded on the tissue manipulation machine 200. In one embodiment, the ID information is sent to an electronic control system 294, and used to automatically determine the process to be executed by the tissue manipulation machine 200. For example, the ID information may contain a serial number which the tissue manipulation machine 200 reads and determines that the complementary device 300 is going to be used to wash and dissociate a fat tissue sample. The tissue manipulation machine 200 may then execute a particular pre-programmed process to perform fat washing and dissociation. In another example, the ID information contains the parameter information of a process. The tissue manipulation machine 200 may read the ID information and execute a protocol using the parameters specified in by the complementary device 300. The information provided by the identification (ID) tag 341 may not be limited to the identification information. For example, an identification (ID) tag 341 may contain a subroutine to be carried out by a tissue manipulation machine. The ID information may also be used to determine how the tissue manipulation machine 200 interacts with a user, for example, to change the user interface, display messages in a certain language, give the user extra flexibility to change process parameters, etc. For example, the tissue manipulation machine 200 may read the ID information from a complementary device 300, determine that the user interface should be shown in Korean, and run a first preprogrammed subroutine. Multiple subroutines, for example, between about 3 and about 10,000, about 3, about 5, about 10, about 20, about 50, about 100, about 200, about 300, about 500, about 800, about 1,000, about 2,000, about 3,000, about 4,000, about 5,000, about 7,000, or about 10,000 subroutines may be pre-loaded onto the tissue manipulation machine 200, for example, onto the electronic control system 294 of the tissue manipulation machine 200. As the term is used herein, a subroutine refers to, but is not limited to, a pre-programmed instruction that controls a sequence of events, including processes, that may be executed by a tissue manipulation machine 200. The subroutines and processes may be updated, for example, through the internet through wired or wireless connection. The ID information may also be used to determine whether the complementary device 300 is authentic, used, or expired.

In one embodiment, the tissue manipulation machine includes a tag reader that reads information from a tag device containing instructions of at least one tissue processing process, information about which processing subroutine to run, or information about a processing subroutine. The tag reader may be configured to access (read and/or write) information on a tag device, which may be attached to a complementary device or separate from a complementary device. Advantages of attaching the tag device to the complementary device may include minimizing risks of running a wrong process using a complementary device.

In the exemplary system shown in FIGS. 1A-1E, the complementary device 300 includes a radio-frequency identification (RFID) tag, and the tissue manipulation machine 200 includes a RFID reader. The tissue manipulation machine may further include a RFID writer to alter or erase the ID information, or to inactivate or burn the RFID tag. The identification (ID) tag system may help prevent reuse or counterfeiting of the complementary device 300, providing a high level of safety and quality control.

It is appreciated that the method disclosed herein of using an ID tag on a complementary device 300 to determine the process to be performed by a tissue manipulation machine 200 is not limited to the particular systems disclosed herein. It is also appreciated that an ID tag described herein is not limited to providing only ID information. An ID tag may provide other information including process, subroutine, and/or product information, and may even retain new information such as whether the complementary device has been used, how many times the complementary device has been used, the time and date in which the complementary device is used, by which machine which the complementary device is used, etc. In one embodiment of the present disclosure, a system for processing clinical samples comprises a single-use complementary device 300 and a tissue manipulation machine 200, wherein the complementary device 300 includes an ID tag 341 that contains information that enables and/or determines the process to be performed by the tissue manipulation machine 200. Such a system may present significant advantages of providing high level of safety, quality control, user experience, and automation, while minimizing potential error. The system may be used in other medical devices, laboratory equipment, industrial equipment and systems, etc.

The electronic control system or controller 294 may include a processor and/or a computer, whether external, built-in, or embedded. The processor may include random access memory (RAM), storage (for example, a hard drive or flash memory), a graphics accelerator, and one or more microcontrollers. The processor may also include RS-232, Universal Serial Bus (USB), Ethernet, high-definition multimedia interface (HDMI), peripheral component interconnect (PCI), peripheral component interconnect express (PCI Express) connectors, and/or any other connectors, an internal bus, and/or an external bus for data transfer known in the art. The processor may be programmed with software, for example, an operating system, which may include one of Linux, Microsoft Windows, and/or Android, and/or firmware to control the tissue manipulation machine 200, for example, the temperature control system 291, the fluid control system 292, the fluid mixing system 293, the user interface 295, and/or the detection feedback system 296. The software may be updated periodically or from time to time. The electronic control system 294 may further include a control unit to supplement the processor. The control unit may comprise a driver, a high current driver for an actuator, a power driver for a heating element, a power driver for a cooling element, a signal conditioning circuit, a digital-to-analog converter (DAC), an analog-to-digital converter (ADC), a pulse modulator, and/or a communication bus. The control unit may be implemented on a printed circuit board (PCB), but it may also be implemented as a discrete circuit without a circuit board, for example, a wire wrap or a point-to-point construction. In one embodiment of the present disclosure, a processor and a control unit are integrated into one computer. In another embodiment of the present disclosure, a system comprises a tissue manipulation machine 200 and an external computer, for example a smart phone, a tablet computer, a laptop computer, or a desktop computer, which may control the tissue manipulation machine 200.

The user interface 295 may comprise at least one device to receive user input, for example, a button switch, a keyboard, a track ball, a mouse, a joystick, a touch screen, a color LCD touch screen, etc., and a transducer to generate a signal, for example, a light emitting device (LED) to generate a light signal, a speaker to generate a sound, a buzzer to generate a buzz, a liquid-crystal display (LCD) to show a message, a built-in LCD display to show a graphic interface (GUI), an external monitor, a touch screen, a color LCD touch screen, etc. In one embodiment of the present disclosure a user interface comprises a touch screen display 210 and a graphical user interface (GUI). The GUI may run as a single, full-screen window and may comprise graphic objects such as a button, a numeric keyboard, a keyboard, a QWERTY keyboard, an input field, a text input field, sliders for user input, and/or graphic objects such as an image, a status bar, a text label, an icon, and an animation for user output. The user interface 295 may prompt an operator (user) to enter a sample ID for a tissue sample to be processed. The sample ID may be uniquely assigned to a patient, a donor or an animal from whom the tissue sample is extracted. The sample ID may be used for traceability and quality control purposes. The user interface 295 may further provide messages, whether textual or graphic, to notify the user of the status, warnings and/or errors of the tissue manipulation machine 200, to prompt the user to perform operating procedures, for example, loading the complementary device 300, loading the tissue sample, and/or unloading the complementary device 300, to prompt the user to select the process to be executed, and/or to ask the user to provide process parameters.

In one embodiment of the present disclosure the electronic control system 294 and/or the user interface 295 may include a computer device, for example, a laptop computer, a smart phone, a tablet computer, a portable computer, a desktop computer, etc., external to the tissue manipulation machine 200.

It is appreciated that embodiments disclosed herein may be applied to many types of tissue samples as defined in the present disclosure. It is also appreciated that the chassis 290, the temperature control system 291, the fluid control system 292, the fluid mixing system 293, the electronic control system 294, the user interface 295, and the detection feedback system 296 disclosed herein may be used in other embodiments and configurations disclosed in the present disclosure.

In another embodiment of the present disclosure, a tissue manipulation system comprises a complementary device, which provides a sterile, preferably single-use, semi-closed or a closed system in which adipose tissues may be collected from a patient, optionally washed, and passed to a re-injection cannula, and a lipotransfer machine, which provides suction, optional tissue washing, and tissue dispensing functions using electronic, mechanical and/or computer control.

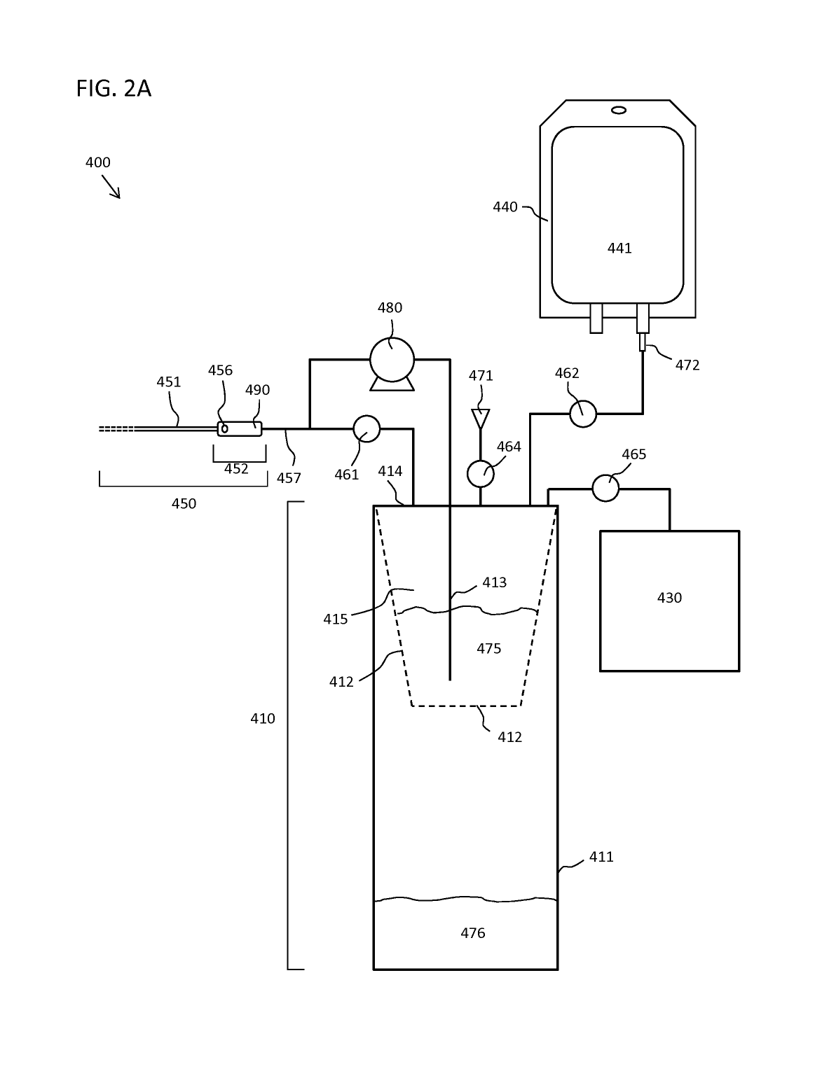

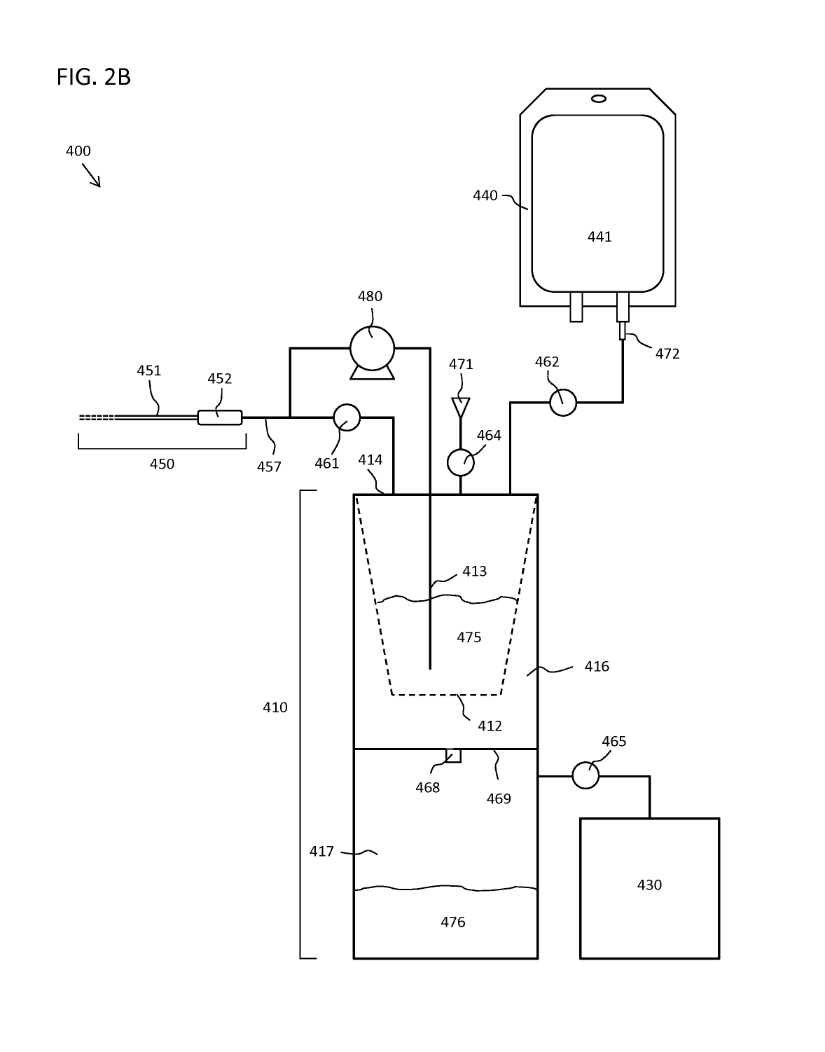

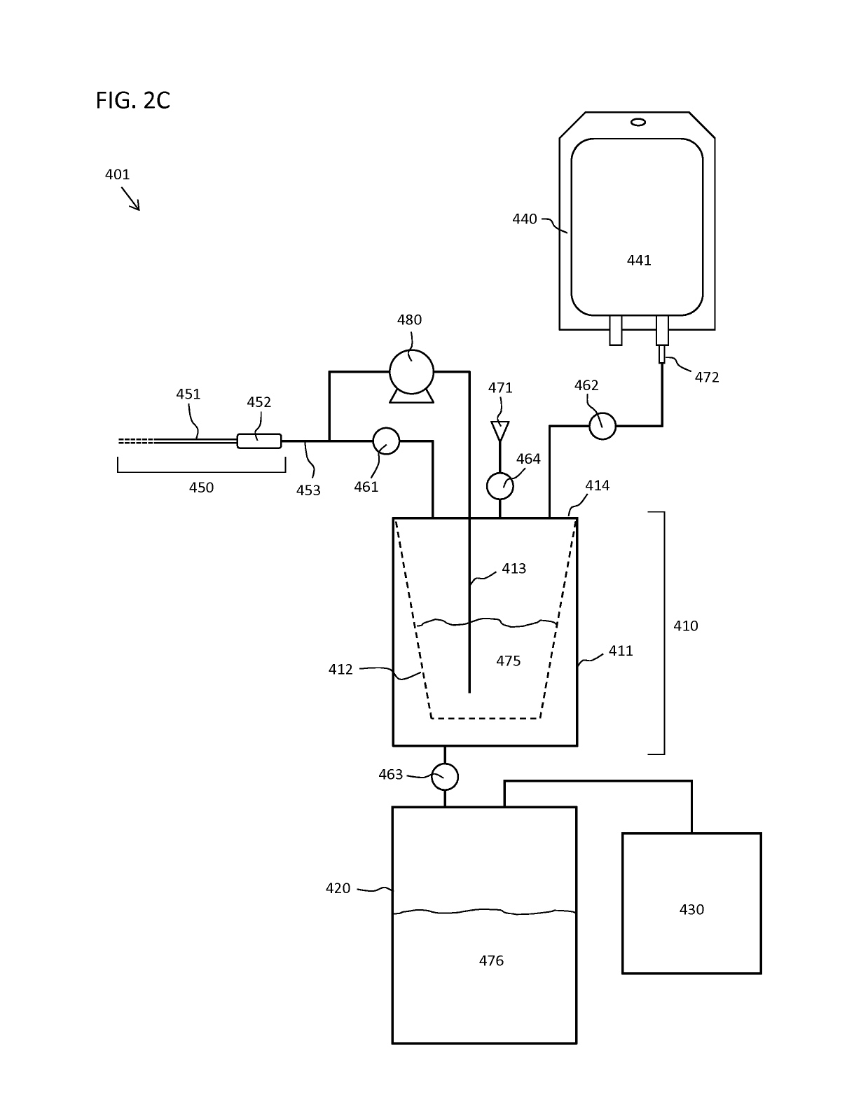

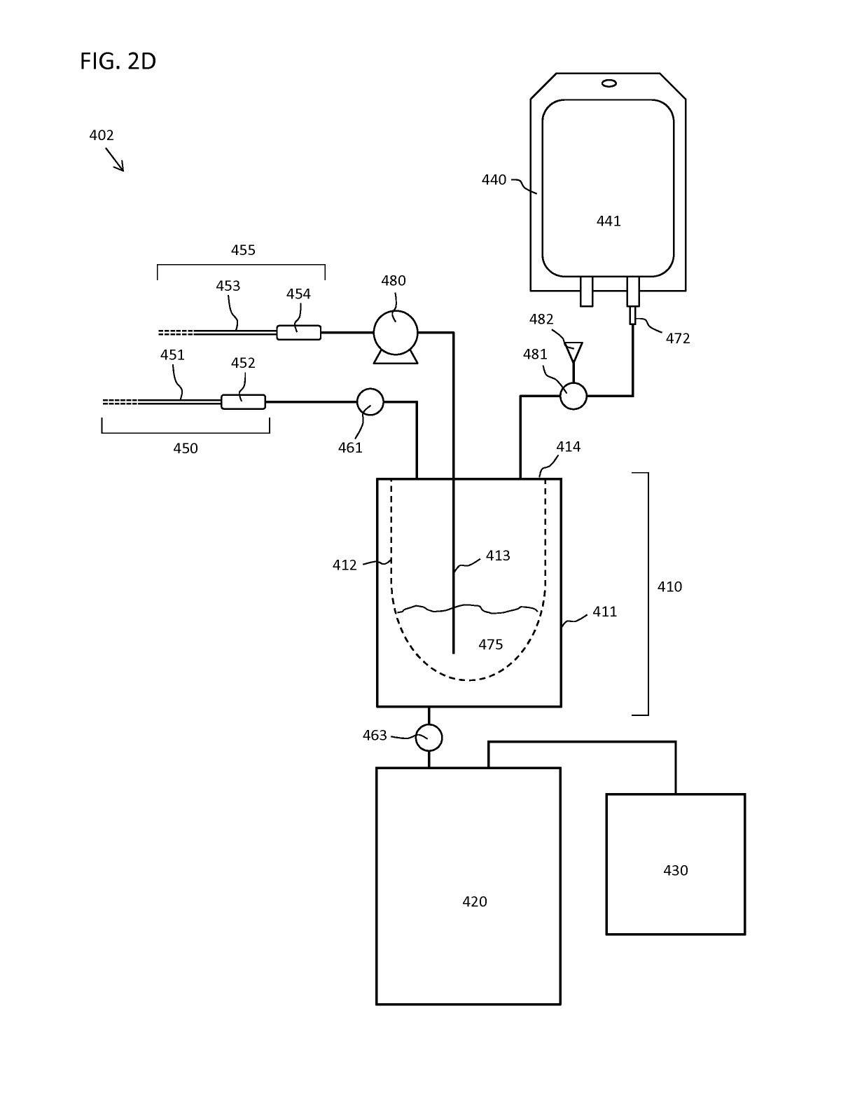

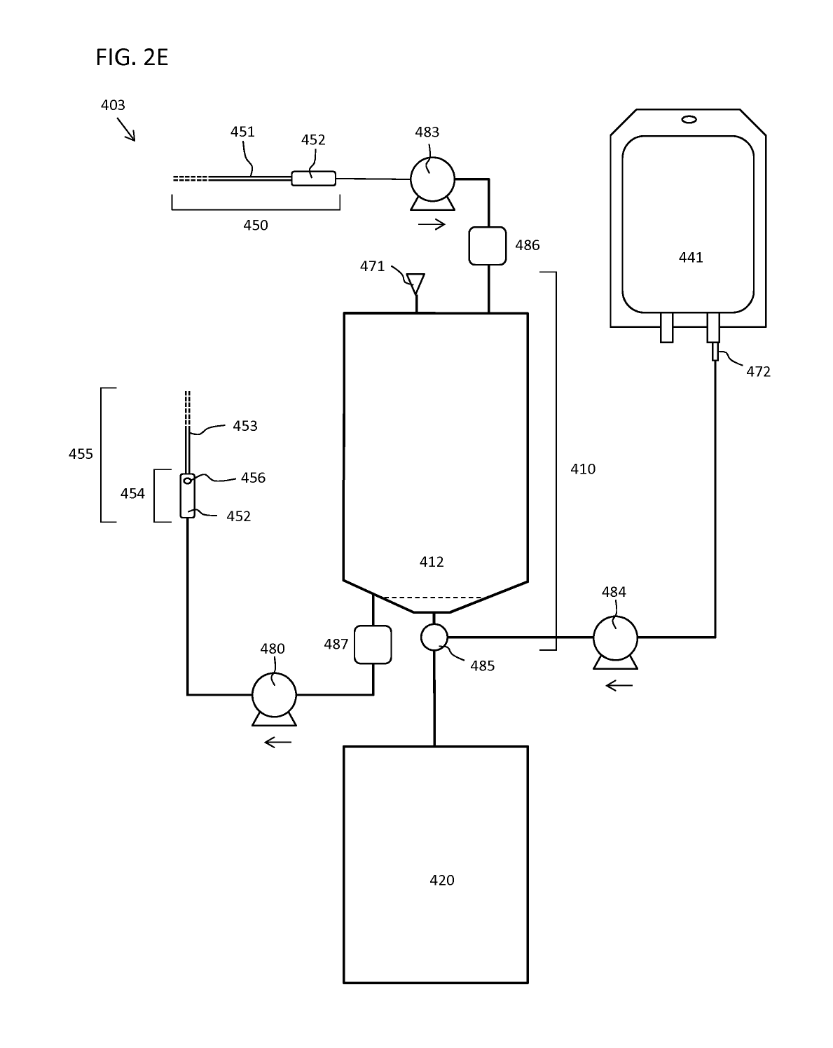

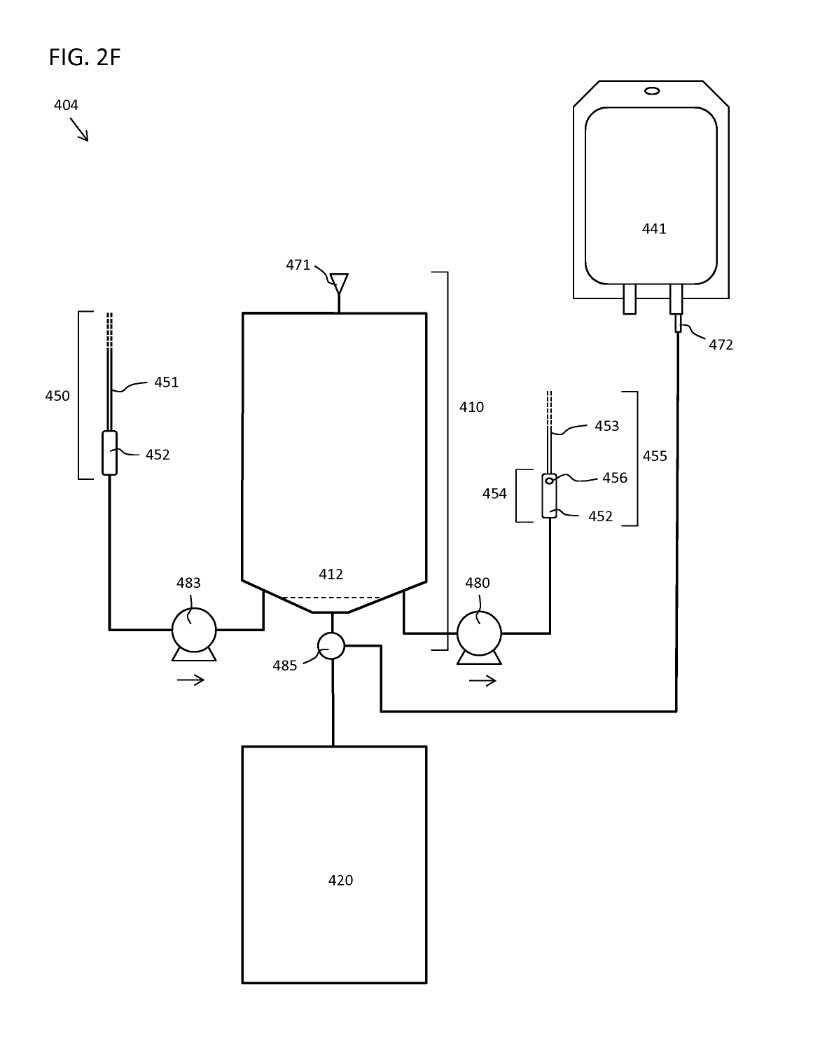

In yet another embodiment of the present disclosure, there is provided a tissue manipulation system, indicated generally at 400 in FIG. 2A. The system comprises a processing unit 410, a tissue extraction and/or injection device, and a tissue pump 480. The tissue extraction and/or injection device may comprise a cannula assembly 450 including a cannula connector 452 and a cannula 451. The cannula connector 452 may be configured to connect to the cannula 451, which may be configured and used to draw adipose tissues from a patient during a liposuction procedure, and/or to inject fat into a patient. The cannula 451 may also be configured and/or used for other purposes. When used for liposuction, adipose tissues are broken into pieces by the cannula 451. The cannula 451 may be a sterile device, for example, a single-use device that is wrapped in a sterile package or a reusable device that can be sterilized or autoclaved. The cannula connector 452 may comprise a cannula handle 490, which makes it easy for a user (for example, a surgeon) to hold the cannula assembly 450 during a procedure. The cannula handle 490 may be ergonomically configured to facilitate a good grip by an operator and reduce operator fatigue. The cannula handle 490 may be configured and arranged for use in a sterile field in an operating room. For example, the cannula handle 490 may be a single use, sterile device. In another example, the cannula handle 490 may be a reusable device that can be autoclaved. In yet another example, the cannula handle 490 may have a sterile wrap that may be replaced after use. The cannula assembly 450 may be connected to the processing unit 410 through a tubing 457 and a suction control valve 461.

The processing unit 410 includes a collection canister 411 and a mesh chamber 415 disposed within the collection canister 411. The collection canister 411 may comprise one or more rigid canisters of varying sizes. The collection canister 411 may also or alternatively comprise of one or more flexible bags. The one or more flexible bags may be supported with an internal or external frame configured to reduce the likelihood of collapse when a vacuum is applied. The collection canister 411 may be sized to hold any desired amount of tissue samples to be collected, as well as waste solutions. The mesh chamber 415 may contain a mesh filter 412 configured to retain tissue pieces and drain fluids, for example, blood, free oil, and tumescent solutions, which may be collected at the bottom of the collection canister 411. The pore size of the mesh filter 412 may be selected based on the tissue type to be manipulated, and/or the bore size of the cannula 451. For example, pore sizes of between about 50 μm and about 400 μm may be used for lipoaspirate samples. Specifically, pore sizes of between about 70 μm and about 300 μm, for example, about 50 μm, about 60 μm, about 70 μm, about 80 μm, about 90 μm, about 100 μm, about 110 μm, about 125 μm, about 150 μm, about 175 μm, about 200 μm, about 250 μm, about 300 μm, about 350 μm, or about 400 μm may be used for lipoaspirate samples. The processing unit 410 may be connected to a vacuum source 430. In one embodiment the vacuum source 430 comprises a vacuum pump and a pressure regulator. The vacuum source 430 may generate a vacuum of between about −0.1 psi and about −14.6 psi with respect to the ambient pressure. Specifically, the vacuum source 430 may generate a vacuum of about −0.2 psi, about −0.5 psi, about −1 psi, about −2 psi, about −3 psi, about −4 psi, about −5 psi, about −6 psi, about −7 psi, about −8 psi, about −9 psi, about −10 psi, about −11 psi, about −12 psi, about −13 psi, or about −14 psi. A vacuum control valve 465 may be used to control and/or regulate the vacuum applied to the processing unit 410. In one embodiment of the present disclosure, the tissue manipulation system 400 may provide accurate control of the suction pressure, which may be regulated by the vacuum source 430, the vacuum control valve 465, and/or the suction control valve 461.

The processing unit 410 may include a vent filter 471, for example a ˜0.2 μm rated membrane filter, a ˜0.45 μm rated polytetrafluoroethylene membrane filter, or another vent filter known in the art, for releasing positive or negative pressures in the collection canister 411 while keeping the space within the processing unit 410 clean and/or sterile. A vent valve 464, for example, a pinch valve or a stopcock valve, may be used to control the pressure release. A stopcock valve 481 may be used to control the flow of rinse solution as well as venting of the collection canister 411 using a vent filter 482, as shown in FIG. 2D. In one embodiment of the present disclosure the vent filter is used to maintain the sterility of an inner space of the processing unit 410. In another embodiment of the present disclosure vent filters are not used.

The processing unit 410 may be connected to a source of rinse solution 441 packaged in a bag 440, for example, a Lactated Ringer's solution (LRS), a saline solution, a normal saline solution, a 0.9% w/v sodium chloride solution, and/or a Ringer's solution packaged in a bag 440. A spike connector 472 may be used to fluidicly connect the rinse solution 441 to the processing unit 410. A rinse solution control element 462, for example, a valve, a pinch valve, or a stopcock valve, may be used to control addition of the rinse solution 441 to the processing unit 410. Alternatively, the rinse solution control element 462 may include a pump, for example, a peristaltic pump or a syringe pump to accurately control the amount of rinse solution 441 added to the processing unit 410. The rinse solution 441 may be added to the collected sample 475 to rinse the sample. Waste solutions 476 may be collected at the bottom of the collection canister 411. In one embodiment, the processing unit 410 includes a mixing mechanism, for example a stir bar or a magnetic stir bar, disposed in the mesh canister 415 in contact with the collected sample 475 to facilitate rinsing the collected sample with the rinse solution 441. In another embodiment, the processing unit 410 is temperature controlled, where the temperature in the mesh chamber 415 is maintained at, for example, between about 20° C. and about 40° C., between about 25° C. and about 37° C., at about 4° C., at about 8° C., at about 12° C., at below about 20° C., at about 22° C., at about 25° C., at about 28° C., at about 30° C., at about 33° C., or at about 37° C. In yet another embodiment, the rinse solution 441 is warmed or chilled to, for example, between about 20° C. and about 40° C., between about 25° C. and about 37° C., at about 4° C., at about 8° C., at about 12° C., at below about 20° C., at about 22° C., about 25° C., about 28° C., about 30° C., about 33° C., or about 37° C.

The processing unit 410 may further include a tissue transfer tube (TTT) 413. The tissue transfer tube 413 contains an opening, which may be positioned close to the bottom of the mesh chamber 415, and is configured to withdraw portions of the collected sample 475 from within the mesh chamber 415. The tissue pump 480 may be configured to transfer the portions of the collected sample 475 through the tissue transfer tube 413 towards the cannula assembly 450. A cannula 451 suitable for injection may be used to inject the portions of the collected sample 475 into a patient. In one embodiment of the present disclosure, the tissue manipulation system 400 provides accurate control over speed of the tissue pump 480. In another embodiment of the present disclosure, the tissue manipulation system 400 provides accurate control over flow rate of the tissue pump 480. Specifically, the tissue manipulation system 400 may provide at least one controlled tissue dispense rate of between about 0.02 ml/sec and about 20 ml/sec, about 0.02 ml/sec, about 0.025 ml/sec, about 0.03 ml/sec, about 0.04 ml/sec, about 0.05 ml/sec, about 0.06 ml/sec, about 0.075 ml/sec, about 0.09 ml/sec, about 0.1 ml/sec, about 0.15 ml/sec, about 0.2 ml/sec, about 0.25 ml/sec, about 0.03 ml/sec, about 0.4 ml/sec, about 0.5 ml/sec, about 0.6 ml/sec, about 0.7 ml/sec, about 0.8 ml/sec, about 1 ml/sec, about 1.5 ml/sec, about 2 ml/sec, about 3 ml/sec, about 5 ml/sec, about 7 ml/sec, about 10 ml/sec, and/or about 20 ml/sec. In another embodiment of the present disclosure, the tissue manipulation system 400 provides accurate control over flow rate of the tissue pump 480. In yet another embodiment of the present disclosure, the tissue pump 480 of the tissue manipulation system 400 provides intermittent and/or pulsed dispensation of the collected tissue sample 475.

In one embodiment of the present disclosure, the tissue manipulation system 400 may be switched between at least two modes, a first mode and a second mode. In the first mode, the tissue pump 480 is turned off (or disarmed), and the suction control valve 461 is armed and may be open. A negative pressure (vacuum) generated from the vacuum source 430 may be applied to the processing unit 410. The first mode may provide suction to the cannula. The tissue manipulation system 400 may be configured to control suction by actuating the fluid control elements included in the system, for example the suction control valve 461, the vacuum control valve 465, and/or the vacuum source 430. The first mode may be used to perform liposuction and to harvest fat tissues (lipoaspirate).

In the second mode, the tissue pump 480 may be armed (and may be turned on and/or activated), and the suction control valve 461 may be closed. Tissue materials of the collected sample 475 may be dispensed at the cannula 451 using a driving force provided by the tissue pump 480. A vent filter 471 may be employed (by opening vent valve 464) to prevent or release negative pressure build up in the processing unit 410. The second mode may provide tissue dispensation at the cannula 451 and may be used for fat injection. The tissue manipulation system 400 may be configured to control the intensity of dispensation by actuating the fluid control elements included in the system, for example, the tissue pump 480. The dispensation of tissues may be continuous, intermittent, or pulsed.

In another embodiment of the present disclosure, the tissue manipulation system 400 may further be switched to a third mode, where a rinse solution 441 may be used to rinse materials collected in the mesh chamber 415. In the third mode, the vent filter 471 may be engaged to prevent positive pressure build up when the rinse solution 441 is introduced into the processing unit 410.

The tissue manipulation system 400 may include a user interface, for example a mechanical switch, a dial knob, a set of buttons, a keyboard, a foot pedal, or a touch screen, to switch between the various modes. The first mode, the second mode, and optionally the third mode enable the tissue manipulation system 400 to provide semi-automated and/or power assisted liposuction, reinjection, and, optionally, fat washing in a semi-closed or closed system.

The tissue manipulation system 400 may include at least one user control element, for example a switch, a button, a dial knob, or a foot pedal, to control suction or dispensation in the first mode or second mode, respectively. The user control element may include a button 456 on the cannula handle 490. The user control element may enable a single user, for example, a plastic surgeon, to control the suction intensity or dispensation speed of tissue manipulation system 400 while performing a procedure.

Conventionally, a plastic surgeon may have to use a syringe and manually control the suction intensity or dispensation speed based on how hard the surgeon pulls or pushes on the plunger of the syringe. The conventional process may put extensive strain on the surgeon's hand, causing hand fatigue, and is subject to operator to operator and procedure to procedure variances. The exemplary tissue manipulation system 400 may provide controlled suction and dispensation in a power assisted and machine controlled manner where the operator only needs to press on the user control element gently, increasing the quality of fat transfer procedure, reducing fatigue of operators, and improving the outcomes. The actuators controlling suction and/or dispensation on the tissue manipulation system 400 may respond to signals from the user control element in a binary manner where the actuation may be switched either on or off, in a discontinuously variable manner where the strength of actuation may be switched to one of the multiple levels of strength, or in a continuously variable manner where the strength of actuation may be adjusted continuous based on the extent to which the user control element is pressed.

In one embodiment of the present disclosure the tissue manipulation system 400 includes a user control element that controls suction and dispensation provided by the system. In another embodiment of the present invention the tissue manipulation system 400 includes a first user control element that controls the suction and a second user control element that controls the dispensation provided by the system.

In another embodiment of the present disclosure, the tissue manipulation system 400 includes a collection canister 411 that is divided by a wall 469 into two compartments, a tissue collection compartment 416 and a waste collection compartment 417 (FIG. 2B). The vacuum source 430 may be connected to the waste collection compartment 417. The tissue collection compartment 416 and the waste collection compartment 417 may be connected by a fluid passage 468, which may include a check valve, for example, a duckbill valve, an umbrella valve, an elastomeric valve, a ball valve, and/or other check valve configurations known in the art, or a filter membrane, configured to allow passage of waste solutions into the waste collection compartment 417 when vacuum is applied. The waste collection compartment 417 may be separated from the tissue collection compartment 416, and may prevent waste solution 476 from mixing with the tissue sample 475.

In yet another embodiment of the present disclosure, a tissue manipulation system 401 includes a tissue collection canister 411 and a waste collection canister 420 (FIG. 2C). The vacuum source 430 may be connected to the waste collection canister 420. The tissue collection canister 411 and waste collection canister 420 may be connected by a fluid passage comprising a valve 463, for example, a pinch valve or a stopcock valve, which may be used control and/or regulate the vacuum supplied to the tissue collection canister 411.

In yet another embodiment of the present disclosure, a tissue manipulation system 402 (FIG. 2D) includes a suction cannula assembly 450 including a suction cannula connector 452 configured to couple to a suction cannula 451, and an injection cannula assembly 455 including an injection cannula connector 454 configured to couple to an injection cannula 453. The tissue manipulation system 402 may provide suction to the suction cannula 451 and may dispense collected tissue through the injection cannula 453. Optionally, a rinse solution 441 may be provided to rinse or wash the collected tissue sample 475. The system 402 may be configured to either provide suction or dispense tissue, but not provide suction and tissue dispensation at the same time. Alternatively, the system 402 may be configured to provide suction at the suction cannula 451 and dispensation of a tissue sample at an injection cannula 453 at the same time. The system 402 may further perform tissue washing using a rinse solution 441 together with suction and/or tissue dispensation. Such a system 402 may enable liposuction and lipo reinjection procedures to be performed at the same time, for example, by two surgeons, significantly increasing the efficiency of lipotransfer procedures.

In yet another embodiment of the present disclosure a processing unit 410 may include a tissue strainer configured to remove large tissue pieces from the lipoaspirate. A tissue strainer may comprise a mesh having pores of about 3 mm, about 4 mm, about 5 mm, about 6 mm, about 7 mm, about 8 mm, about 1 cm, or larger. A tissue strainer may comprise a passages of about 3 mm, about 4 mm, about 5 mm, about 6 mm, about 7 mm, about 8 mm, about 1 cm, or larger. A tissue strainer may comprise slots of about 2 mm, about 2.5 mm, about 3 mm, about 4 mm, about 5 mm, about 6 mm, about 7 mm, about 8 mm, about 10 mm, about 12 mm, or about 15 mm wide, configured to retain tissue pieces that may be too large to smoothly pass through the output tissue pump and the re-injection cannula.

In yet another embodiment of the present disclosure, a tissue manipulation system comprises a tissue manipulation machine and a complementary device 403, schematically shown in FIG. 2E. The complementary device 403 includes a processing unit 410, an output tissue pump 480, an input tissue pump 483, and a waste collection compartment 420. The processing unit 410 includes at least one tissue inlet, at least one tissue outlet, a waste solution outlet, and optionally a rinse solution inlet. The tissue inlet is fluidicly connected to the input tissue pump 483, which is fluidicly connected to a suction cannula 451, and which is configured to draw adipose tissues as lipoaspirate from a patient and deposit the lipoaspirate into the processing unit 410. The input tissue pump 483 may comprise a syringe pump including at least one syringe having a volume of about 10 ml, about 20 ml, about 30 ml, about 60 ml, about 100 ml, about 150 ml, or about 200 ml. The processing unit 410 may comprise a pouch, a bag, a flexible compartment made of, for example, PVC sheets or polyurethane (PU) sheets, a canister or a container, and may contain a mesh 412 configured to retain the adipose tissues and drain waste fluids, for example blood, tumescent solutions, and/or other body fluids. Specifically, the mesh 412 is configured to allow adipose tissues to pass from the tissue inlet to the tissue outlet, and not from the tissue inlet to the waste solution outlet. The waste solution outlet is fluidicly connected to the waste collection compartment 420, which may comprise a pouch, a bag, a canister or a container, for example. The processing unit 410 may further be fluidicly connected to a source of rinse solution, for example a Lactated Ringer's solution (LRS), a saline solution, a normal saline solution 441, a 0.9% w/v sodium chloride solution, a Ringer's solution, using a connector 472, for example, a luer connector, a catheter connector, or a spike connector. The output tissue pump 480 is fluidicly connected to the processing unit 410 at the tissue outlet, and configured to transfer adipose tissue from the processing unit 410 towards an injection cannula 453. The output tissue pump 480 may comprise a syringe pump including at least one syringe having a volume of about 1.5 ml, about 2 ml, about 2.5 ml, about 3 ml, about 5 ml, about 7 ml, about 10 ml, about 12 ml, about 15 ml, or about 20 ml. The processing unit 410 may further comprise at least one valve, for example, a stopcock valve 485, configured to control the flow of the rinse solution and/or the waste solution. For example, the stopcock 485 may connect the processing unit 410 to the waste collection compartment 420 for draining waste solution, or may connect the processing unit 410 to the rinse solution 441 to introduce the rinse solution for washing the tissues. The processing unit 410 may include a vent filter 471 to release extra air pressure that may build up in the processing unit 410. The complementary device 403 may optionally include a rinse solution pump 484 to provide accurate control of the rinse solution flow. A tissue strainer 486 may be included between the input tissue pump 483 and the processing unit 410 to filter out large tissue pieces that is not suitable for re-injection. A tissue strainer 487 may also be included between the tissue outlet of the processing unit 410 and the output tissue pump 480. The tissue outlet of the processing unit 410 may be configured to be at lower positions than the deposited adipose tissues. Whereas the tissue inlet of the processing unit 410 may be configured at the top of the processing unit as shown in FIG. 2E, it may also be configured at the bottom of the processing unit as shown in FIG. 2F. The positions, shapes and configurations of the complementary device of the present disclosure are not limited to the embodiments shown in the figures herein.

In yet another embodiment of the present disclosure, a tissue manipulation system for lipotransfer includes a modulator disposed between the output tissue pump and the injection cannula, and configured to modulate the tissue flow passing through the injection cannula. The modulator may improve the control of tissue flow at the injection cannula, and may allow for discrete dispense of the tissue. This may be very desirable as lipoaspirate tissues includes tissue pieces that are not homogeneous, and dispensing small volumes of lipoaspirate with precision may be challenging when performed manually. In one embodiment, the modulator comprises a tissue pump. The modulator may serve as a second stage pump to the primary output tissue pump. In another embodiment, the modulator comprises a positive displacement pump. In yet another embodiment, the modulator is housed in an injection cannula connector, which may serve as a hand piece connected to an injection cannula. In yet another embodiment, the modulator comprises a syringe pump including a syringe of about 0.5 ml, about 1 ml, about 1.5 ml, about 2 ml, about 2.5 ml, about 3 ml, about 5 ml, or about 10 ml. In yet another embodiment, the modulator comprises a flexible conduit having an inlet and an outlet, a first check valve, for example a duckbill valve, a cross slit valve or a dome valve, at the inlet end of the flexible conduit, and optionally a second check valve at the outlet end of the flexible conduit. The flexible conduit may be squeezed and relaxed to output a pulse of tissues at the outlet end. The modulator may be housed in an injection cannula connector, also referred to as a cannula handle or a cannula hand piece, which may serve as a hand piece for holding the injection cannula, and the outlet end of the conduit may be in close proximity to the injection cannula, improving the control and precision of tissue dispensing at the injection cannula. In yet another embodiment, a modulator housed in a cannula hand piece includes passive configurations to allow for manual activation of the modulator. For example, the modulator may be a syringe that may be refilled with the tissues transferred from a processing unit using a tissue pump. The modulator may include mechanisms housed in the cannula hand piece and configured to manually inject a pre-determined volume from the syringe into the injection cannula, as disclosed in U.S. Pat. No. 8,801,659 B2, U.S. Pat. No. 8,632,498 B2, U.S. Pat. No. 7,632,251 B2 and U.S. Pat. No. 8,523,825. For another example, a modulator comprising a flexible conduit and at least one check valve housed in a cannula hand piece may include a push button, for example the button 456 in FIG. 2. The button may be configured to apply pressure on the flexible conduit, thereby dispensing a pulse of tissues through the injection cannula upon pressing the button. The modulator may alternatively include actuators housed in the cannula hand piece configured to drive a pre-determined volume of tissue through the injection cannula. The actuators may be battery powered, electrically powered, mechanically powered and/or pneumatically powered. In yet another embodiment, the modulator is configured to dispense from the injection cannula a sequence of discrete bits of tissues, where each bit may have a pre-determined nominal volume of about 10 μl, about 15 μl, about 20 μl, about 25 μl, about 30 μl, about 40 μl, about 50 μl, about 60 μl, about 75 μl, about 90 μl, about 100 μl, about 125 μl, about 150 μl, about 175 μl, about 200 μl, about 250 μl, about 300 μl, about 400 μl, or about 500 μl. The modulator and the output tissue pump may be configured to be synchronized to generate discrete bits of tissue output. For example, the modulator and the output tissue pump may be both actuate to transfer a volume of lipoaspirate from the processing unit towards the injection cannula, upon receiving a signal from a foot pedal or a button on the cannula hand piece. The modulator may also be configured to dispense tissues at more discrete volumes. For example, the output tissue pump may pump a pre-defined amount of tissues towards the modulator, and the modulator may pulse to intensify the pressure used to squeeze out the initial volume of the pre-defined amount of tissues. One of the advantages of dispensing precise and small pulses of volumes of tissues may be to enable in a semi-automatic manner the Coleman technique, which has been shown to increase graft retention and improve lipotransfer outcomes.

In yet another embodiment of the present disclosure, a lipotransfer method includes performing liposuction, tissue washing, and re-injection steps using a tissue manipulation system disclosed herein, for example, a tissue manipulation system represented schematically in FIG. 2E. Liposuction is performed on a patient using a suction cannula 451. Extracted adipose tissues (lipoaspirate) are suctioned and collected in a processing unit 410. A rinse solution is introduced to the processing unit 410 to rinse the lipoaspirate tissues. Mixing, for example, massaging or rocking actions, may be applied to the processing unit 410 to wash the lipoaspirate tissues. Blood, tumescent solutions and other waste fluids are then drained into a waste container 420. The washing step comprising introducing a rinse solution, optionally mix the rinse solution with the tissues to be washed, and draining the waste fluids, may be repeated and performed multiple times, for example twice, three times, four times, or about five times. The washed tissues are pumped out of the processing unit 410 using at least one output tissue pump 480, and injected back to the patient via an injection cannula, preferably in multiple small volumes. The tissue manipulation machine of the tissue manipulation system provides electronic, computer, mechanical, and/or pneumatic control and actuation to achieve automated or semi-automated lipotransfer processes.

In the present disclosure a rinse solution may comprise, and is not limited to, Lactated Ringer's solution (LRS), a saline solution, a normal saline solution 441, a 0.9% w/v sodium chloride solution, Ringer's solution, Hartmann's solution, a compound sodium lactate (CSL) solution, a phosphate buffered saline solution, a Hank's balanced salt solution, a cell culture medium, or other solutions known in the art suitable for human injection, animal injection, or cell culture.

In the present disclosure a suction cannula (for example the suction cannula 451 in FIG. 2) may comprise a cannula used in the field of liposuction, for example a cobra bibevel cannula, a cobra round tip cannula, a mercedes cannula, a pyramid cannula, a standard cannula, a powered cannula, a Stevens speed cannula, etc. The inner diameter of the suction cannula may be between 1.5 mm and 6 mm, between 2.5 mm and 4.5 mm, or more specifically about 3 mm or about 4 mm.

In the present disclosure an injection cannula (for example the cannula 453 in FIG. 2D) may be between gauge 8 and gauge 24, or more specifically between gauge 12 and gauge 20. In one embodiment of the present disclosure the injection cannula is between gauge 14 and gauge 18. The injection cannula may have a round tip, an oval shaped opening, a spoon tip opening, and/or a J.W. Little type opening. It may also be straight or curved.

In the present disclosure a vent filter (for example the vent filter 471 and the vent filter 482 in FIG. 2D) may comprise, and is not limited to, a membrane filter rated for about 0.1 μm, about 0.15 μm, about 0.2 μm, about 0.22 μm, about 0.25 μm, about 0.3 μm, about 0.4 μm, about 0.45 μm, about 0.5 μm, about 0.6 μm, about 0.8 μm, about 1 μm, about 1.5 μm, or about 2 μm, for example a 0.22 μm rated cellulose acetate (CA) membrane filter, a 0.45 μm rated polytetrafluoroethylene (PTFE) membrane filter, etc. For sterile applications, small pore ratings may be preferred.

In one embodiment of the present disclosure a tissue pump (for example, the tissue pump 480 and/or the tissue pump 483 in FIG. 2E) may comprise a positive displacement pump, for example, a reciprocating pump, a rotary lobe pump, a progressive cavity pump, a rotary gear pump, a piston pump, a plunger pump, a diaphragm pump, a screw pump, a gear pump, a rotary vane pump, a regenerative (peripheral) pump, a peristaltic pump, a rope pump, a flexible impeller pump, and a syringe pump. In one embodiment of the present disclosure the tissue pump comprises a syringe pump 500, 510, as shown in FIGS. 3A and 3B. In one example (FIG. 3A), the tissue pump 500 comprises a syringe 501 connected to a stopcock 502. Fluids and/or tissue samples may be driven from the inlet 503 of the pump 500 towards the outlet 504 by first turning the stopcock 502 to fluidicly connect the syringe 501 to the inlet 503, pulling the syringe plunger 505 to fill the syringe 501, turning the stopcock 502 to fluidicly connect the syringe 501 to the outlet 504, and then pushing the plunger 505 to empty the syringe 501. In another example (FIG. 3B), the tissue pump 510 comprises a syringe 511 connected to a first check valve 512 and a second check valve 513. The check valves included may be a duckbill valve, a cross slit valve, a dome valve, or any other valve known in the art suitable for controlling tissue flow. In one embodiment of the present disclosure, the check valves 512, 513 comprise duckbill valves. Fluids and/or tissue samples may be driven from the inlet 514 of the pump 510 towards the outlet 515 by first pulling the syringe plunger 516 to draw fluids into the syringe 511 through check valve 512, and then pushing the plunger 516 to empty the syringe 511 through check valve 513. The syringe pump 500, 510 may be actuated using actuators. For example, a rotational actuator may be used to drive the stopcock 502, and a linear actuator may be used to push and/or pull the plunger of the syringe 501, 511. The syringe included in a syringe pump may have a volume of about 0.5 ml, about 1 ml, about 1.5 ml, about 2 ml, about 2.5 ml, about 3 ml, about 4 ml, about 5 ml, about 7 ml, about 10 ml, about 12 ml, about 15 ml, about 20 ml, about 25 ml, about 30 ml, about 40 ml, about 50 ml, about 60 ml, about 75 ml, about 90 ml, about 100 ml, about 125 ml, about 150 ml, about 200 ml, or about 300 ml.

In another embodiment of the present disclosure the tissue pump 480 comprises a two syringe pumps (FIG. 3C). Because a syringe pump (500 or 510) may pump in cycles including a pull phase and a push phase, a syringe pump may not provide continuous fluid outputs. Two syringe pumps may be fluidicly connected to a common inlet 527 and a common outlet 528 to provide continuous and/or uninterrupted operation. While the first syringe 525 is providing fluids to the outlet 528, the second syringe 526 may be drawing fluids from the inlet 527, and vice versa. Check valves 521, 522, 523, 524 may control the flow of fluid from the common inlet 527 to the common outlet 528. Combining the two syringe pumps, the tissue pump 480 may operate continuously without interruption. When used as an input tissue pump, this configuration may provide continuous suction. When used as an output tissue pump, this configuration may provide continuous dispensing of tissues, or uninterrupted sequence of bits of tissues.

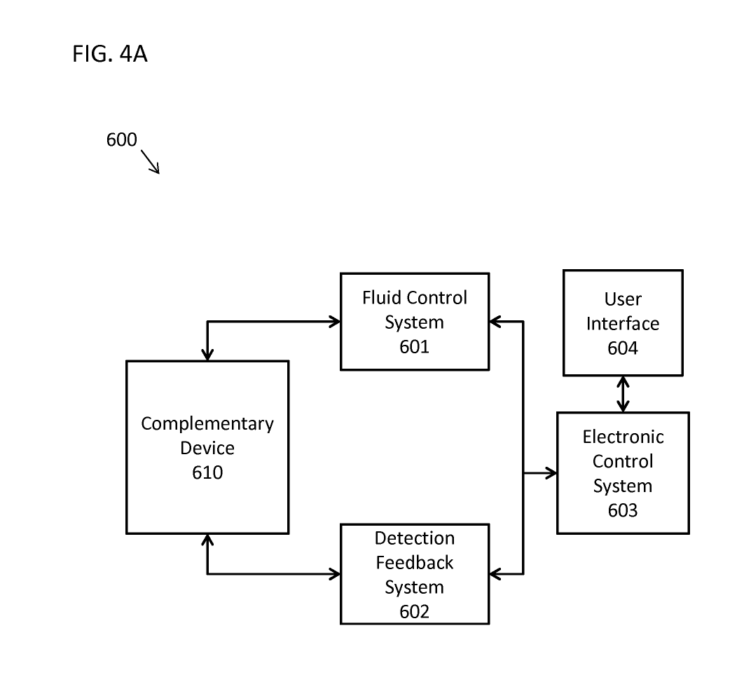

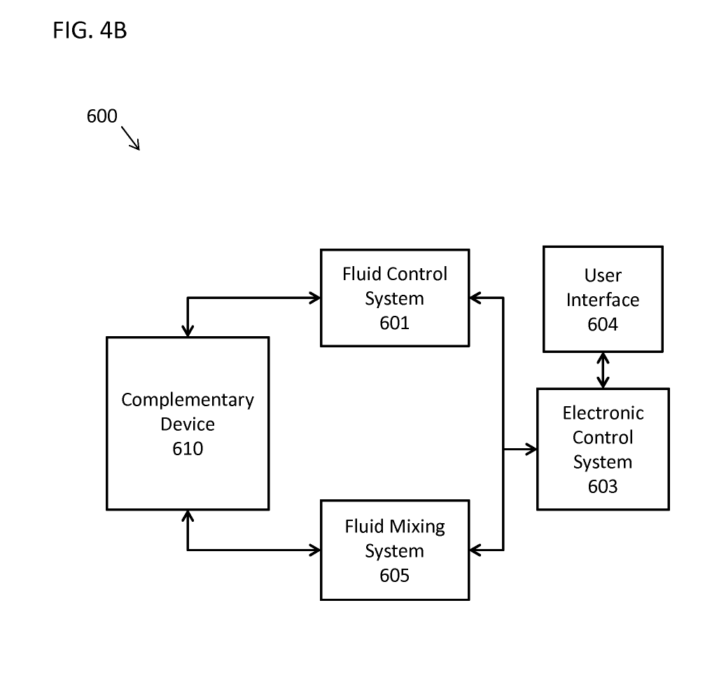

In one embodiment of the present disclosure, a tissue manipulation system 600 (FIG. 4A) may comprise a complementary device, which may include a cannula assembly 450, a processing unit 410, a tissue pump 480, and/or a tissue manipulation machine, which may include a vacuum source 430 (FIGS. 2A, 2B, 2C, 2D). In yet another embodiment of the present disclosure, the tissue manipulation system includes a complementary device, which is sterile and a semi-closed system, and a tissue manipulation machine, which provides electronic and/or computer control and actuation. An exemplary block diagram of the tissue manipulation system 600 is shown in FIG. 4A. The complementary device 610 may be a sterile, single-use and closed device. The complementary device 610 may also include a single-use sterile component and a reusable component that can be autoclaved, for example, a metal cannula. The tissue manipulation machine may further comprise a fluid control system 601 comprising actuators to actuate the fluid pumps, a tissue pump, a modulator, and/or valves contained in the complementary device 610. For example, the tissue manipulation machine may include an actuator to drive a syringe on a complementary device 610, and an actuator comprising a drive hub and a motor or a stepper motor to drive a stopcock valve. The tissue manipulation machine may further comprise a detection feedback system 602 to monitor the status of the machine, the status of the complementary device, and/or the status of the tissue manipulation system. The tissue manipulation machine may further comprise an electronic control system 603 and a user interface 604 to provide electronic control and/or computerized control of the machine. In one embodiment of the present disclosure, a tissue manipulation system comprises at least one of the detection feedback system 602, the electronic control system 603, and the user interface 604. The tissue manipulation machine may further comprise a fluid mixing system 605 (FIG. 4B), for example comprising rollers, massaging mechanism, or another mechanism disclosed in the present disclosure, to mix the rinse solution with the tissue sample to achieve efficient washing.

The user interface of the presently disclosed tissue manipulation system may comprise a sensor configured to receive a signal from a foot pedal or a button on a suction cannula hand piece. The sensor may transduce a pressure signal or an intensity signal from the user to a machine signal, for example an electrical signal or a mechanical signal, which informs the electronic control system of the tissue manipulation machine to apply suction to the suction cannula. The suction applied may be configured to correspond to or be proportional to the pressure or intensity detected by the sensor. A sensor may also be configured to receive a signal from a foot pedal or a button on the injection cannula hand piece. The sensor may detect a signal, which may contain intensity and/or duration information from the user. The tissue manipulation machine then translates the signal to generate a tissue dispensing output, whose dispense rate may correspond to the intensity signal and whose duration may be correspond to the duration signal. In one embodiment of the present disclosure, a tissue manipulation system configured for lipotransfer may dispense a small volume bit of lipoaspirate tissues about every 0.15 seconds, about every 0.2 seconds, about every 0.25 seconds, about every 0.3 seconds, about every 0.4 seconds, about every 0.5 seconds, about every 0.6 seconds, about every 0.7 seconds, about every 0.8 seconds, about every 0.9 seconds, about every second, about every 1.1 seconds, about every 1.25 seconds, about every 1.5 seconds, about every 1.75 seconds, and/or about every 2 seconds. The tissue manipulation system for lipotransfer may be configured to dispense a sequence of small volumes of tissues at a frequency of between about 0 Hz and about 6 Hz, or more specifically between about 0.5 Hz and about 4 Hz.

In yet another embodiment of the present disclosure, the tissue manipulation system 400, 401, 402 may include a tissue extraction device configured to perform power assisted liposuction (PAL), ultrasound-assisted liposuction, waterjet-assisted liposuction, laser liposuction or other liposuction methods known in the art.

In yet another embodiment of the present disclosure, a tissue manipulation system 400 may be used to perform a lipotransfer procedure, including liposuction, fat reinjection, and, optionally, fat washing procedures on a patient, wherein the tissue manipulation system provides a vacuum suction for liposuction and a machine-powered fat dispensation for fat reinjection. In yet another embodiment of the present disclosure, a tissue manipulation system is used to perform liposuction on one portion of a patient to collect a lipoaspirate, optionally wash the lipoaspirate, and reinject the washed lipoaspirate on a different portion of the patient, thereby achieving lipotransfer on the patient.

### Example 1

### Heating of Fluids in a Tissue Processing System

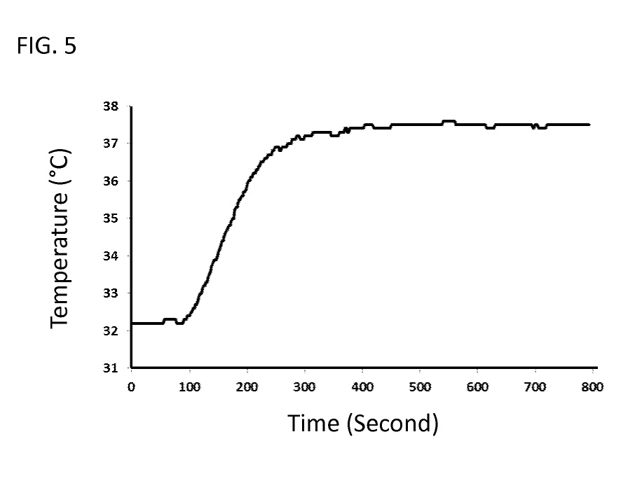

This example show how a tissue processing system comprising a tissue manipulation machine and a complementary device shown in FIGS. 1A and 1C, respectively, may be used to rapidly heat up fluids to a target temperature and maintain the temperature in a narrow range. The complementary device made of two sheets of PVC includes a sample processing compartment of about 16 cm×11 cm. 70 ml of water is loaded in the sample processing compartment, which is in contact with a heating plate. A roller moving at about 10 cm/sec presses on the sample processing compartment to mix the fluids inside at a frequency of about 0.5 Hz. A temperature probe is used to measure the water temperature inside the sample processing compartment and a temperature logger is used record the temperature. A target temperature is set to 37.5° C. in this example. As shown in FIG. 5, the temperature of the water is heated from 32° C. to 37° C., which is 0.5° C. within the target temperature, within about 160 seconds. Within the next 60 seconds the target temperature of 37.5° C. is reached without any overshoot. The temperature may be maintained within positive or negative 0.1° C. from the target temperature of 37.5° C. for 10 minutes.

This example shows that the tissue processing system disclosed in the present disclosure may be configured to rapidly heat a sample from room temperature (about 25° C.) to a target temperature, for example, an optimum temperature for tissue dissociation, an optimum temperature for enzyme digestion, 37° C., etc., within about 500 seconds, about 400 seconds, about 300 seconds, about 250 seconds, about 200 seconds, about 180 seconds, about 150 seconds, about 120 seconds, about 100 seconds, about 90 seconds, about 80 seconds, about 70 seconds, about 60 seconds, about 50 seconds, about 45 seconds, about 40 seconds, about 35 seconds, about 30 seconds, about 25 seconds, or about 20 seconds, without over heating (temperature overshoot) of greater than 2° C., 1.5° C., 1.2° C., 1° C., 0.8° C., 7° C., 0.6° C., 0.5° C., 0.4° C., 0.3° C., 0.2° C., or 0.1° C., and maintain the temperature within ±1° C., ±0.8° C., ±0.6° C., ±0.5° C., ±0.4° C., ±0.3° C., ±0.2° C., or ±0.1° C. with respect to a target temperature, wherein the sample has a volume of between about 1 ml and about 500 ml, for example about 1 ml, about 1.5 ml, about 2 ml, about 3 ml, about 5 ml, about 7 ml, about 10 ml, about 12 ml, about 15 ml, about 20 ml, about 25 ml, about 30 ml, about 35 ml, about 40 ml, about 45 ml, about 50 ml, about 60 ml, about 70 ml, about 80 ml, about 90 ml, about 100 ml, about 120 ml, about 150 ml, about 200 ml, about 250 ml, about 300 ml, about 400 ml, or about 500 ml. The heating element in the temperature control system may be capable of delivering heat at a power of about 1,000 W, about 800 W, about 600 W, about 500 W, about 400 W, about 300 W, about 250 W, about 200 W, about 180 W, about 150 W, about 125 W, about 100 W, about 75 W, about 60 W, about 50 W, about 40 W, about 30 W, about 25 W, about 20 W, about 15 W, or about 10 W. The heating element may be modulated to deliver heat at large range of power, for example between 5 W and 500 W, between 10 W and 1,000 W, between 2 W and 200 W, between 1 W and 100 W, between 3 W and 300 W, between 1 W and 50 W, between 0.3 W and 30 W, etc.

### Example 2

### Isolating Stromal Vascular Fraction (SVF) from Lipoaspirate Samples Using an Automated Tissue Manipulation System

An automated tissue manipulation system 100 comprising a tissue processing machine 200 and a sterile, single-use complementary device 300, shown in FIG. 1A and FIG. 1C, respectively, and disclosed in this present disclosure, is configured to extract stromal vascular fraction (SVF), which may include fibroblasts, smooth muscle cells, endothelial cells, endothelial progenitor cells (EPC), preadipocytes, vascular progenitor cells, hematopoietic progenitor cells, mesenchymal stromal cells, mesenchymal stem cells, hematopoietic stem cells, pericytes, and/or supra-adventicial cells, from fat tissues. The system is configured to process between about 15 ml and about 60 ml of lipoaspirate and/or minced fat tissue from human or animal sources. The system is further configured to perform tissue washing, enzymatic digestion, SVF/adipocyte separation, and debris removal functions automatically using computer control. Lactated Ringers Solution (LRS) in a 500 ml bag is used as the rinsing solution. 100 mg of collagenase NB 4 Standard Grade (SERVA, Cat. No. 17454) dissolved in 10 ml of LRS is used as the dissociation solution. The total processing time is configured to about 50 minutes.

Four samples of fresh human lipoaspirate from different consented donors were processed using the system disclosed herein within 12 hours of liposuction. Between 40 ml and 60 ml of each sample were loaded into the system for processing. After processing, the output is collected automatically in a 60 ml syringe. All output volumes are measured to be around 56 ml to 59 ml. Each output solution is mixed with an equal volume of a culture medium solution containing about 10% of fetal bovine serum and centrifuged at 1200 g for 10 minutes at room temperature. The supernatant is then removed and the cells are resuspended in the culture medium. This solution is enumerated for nucleated cell count and viability using an automatic cell counter (ADAM MC, NanoEnTek Inc., Korea).

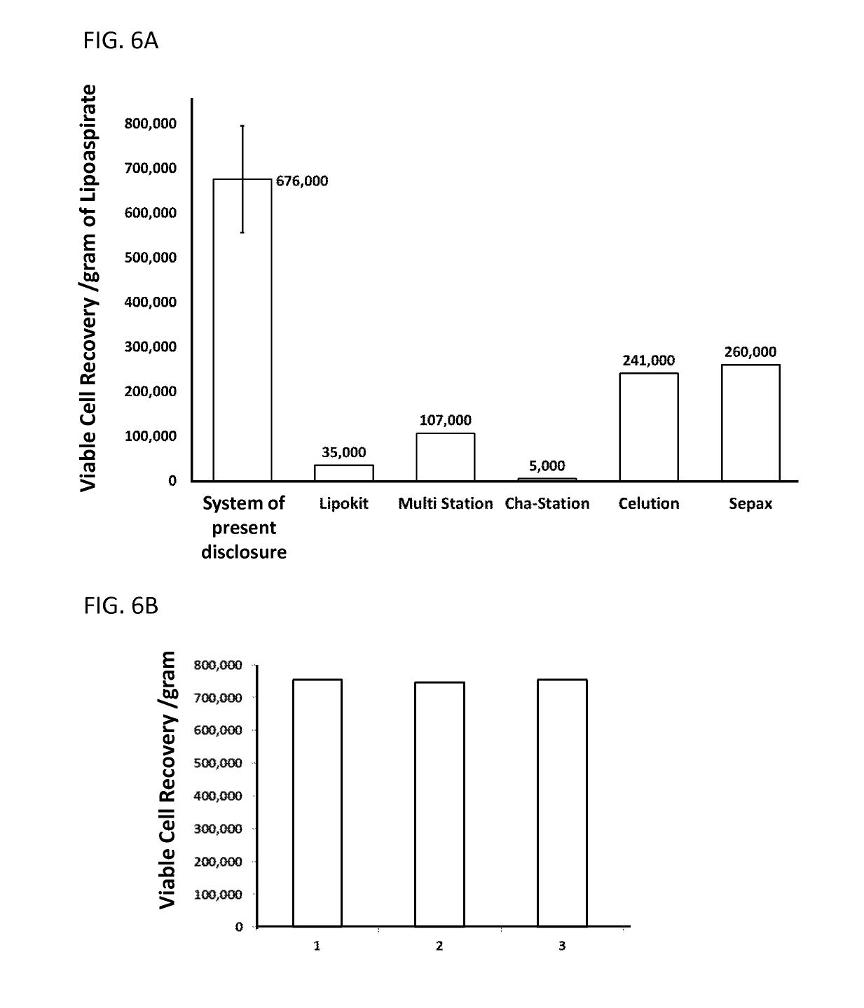

The results are shown in FIG. 6A. FIG. 6A shows the viable cell recovery calculated as the number of viable nucleated cells recovered from a gram of lipoaspirate processed. The average viable cell recovery using the system disclosed herein is plotted alongside the viable cell recovery performance of five other SVF processing systems known in the literature, PNC Multi Station, CHA Biotech Cha-Station, Cytori Celution 800/CRS System, Medi-Khan Lipokit with MaxStem, and Biosafe Sepax (Aronowitz J A, Ellenhorn J D, “Adipose stromal vascular fraction isolation: a head-to-head comparison of four commercial cell separation systems” Plast Reconstr Surg. 2013 December; 132(6): 932e-9e.; Güven S, Karagianni M, Schwalbe M, et. al., “Validation of an automated procedure to isolate human adipose tissue-derived cells by using the Sepax technology” Tissue Eng Part C Methods. 2012 August; 18(8):575-82). Note that the five other SVF processing systems provide highly variable viable nucleated cell recoveries depending on the method and system used to process adipose tissues. The average viable nucleated cell recovery ranges from about 5,000 viable cells per gram of lipoaspirate (Cha-station) to about 260,000 viable cells per gram of lipoaspirate (Sepax), a difference of about 50×. Each individual system of the five systems known in the literature also results in a wide and inconsistent range of viable cell recovery according to the references. In contrast, the system disclosed herein produced a consistent viable cell recovery of between about 500,000 cells per gram of lipoaspirate to about 800,000 cells per gram of lipoaspirate, reflecting the sample to sample variation, with an average of about 676,000 viable cells per gram of lipoaspirate and a standard deviation (represented by the error bar) of about 119,000 cells per gram of lipoaspirate. The inter sample coefficient of variance of viable cell recovery, defined as the standard deviation of viable cell recovered per gram of fat tissue processed, divided by the average of viable cell recovered per gram of fat tissue processed, is about 17.6%. The minimum viable cell recovery of the system disclosed herein (about 500,000 cells/g) is about 2× as much as the average results from Cytori Celution 800/CRS System and Biosafe Sepax, while the average viable cell recovery of the system disclosed herein (about 676,000 cells/g) is about 2.6× as much as the average results from Cytori Celution 800/CRS System and Biosafe Sepax system. The viability of the SVF generated using the system disclosed herein is greater than about 80%, averaging about 85%.

In the next experiment three automated tissue manipulation system 100 were used to process three aliquots of 45 ml of lipoaspirate samples collected from the same liposuction procedure from a donor. 5 mg of Liberase™ (Roche 05401119001) reconstituted in 6 ml of LRS was used as the dissociation solution for each system. After processing, which took about 45 minutes, the cells were collected and neutralized with the same volume of a culture medium containing 12% fetal bovine serum, and centrifuged at 1200 RCF for 10 min. The supernatant was then removed and the cell pellet was resuspended in the culture medium. An automatic cell counter (ADAM MC, NanoEnTek Inc., Korea) was used to enumerate the nucleated cells and measure their viability. Note that autologous serum may be used to neutralize the enzyme instead.

The results are shown in FIG. 6B. The three systems extracted 754,000, 745,000, and 753,000 viable cells from a gram of lipoaspirate respectively. The inter-system intra-sample coefficient of variance on viable cell recovery, defined as the standard deviation of viable cell recovered per gram of fat tissue processed among the systems, divided by the average of viable cell recovered per gram of fat tissue processed, is about 0.6%, showing that the automated system disclosed herein is capable of achieving remarkable reproducibility and run-to-run consistency. Such level of reproducibility may be very difficult to achieve using manual methods considering that an operator may perform the same protocol not exactly the same way every time, and different operators at different laboratories may perform the same protocol even more differently. Such level of reproducibility also has not been demonstrated by any published systems that the applicant is aware of. The high system to system reproducibility may be attributed to the precise computer-controlled temperature control system, fluid control system, and/or fluid mixing system. This high system to system reproducibility ensures that the best possible results and outcomes may be delivered every time regardless of whom the operator is and where the laboratory is. In one embodiment of the present disclosure, a system is configured to achieve an inter-system intra-sample coefficient of variance on viable cell recovery of smaller than 5%, smaller than 4%, smaller than 3%, smaller than 2%, or smaller than 1%. In another embodiment of the present disclosure, a system is configured to achieve an intra-sample variance of within about 5%, about 4%, about 3%, about 2%, about 1%, or about 0.5%.

The system disclosed herein may be capable of recovering between about 500,000 and about 1,000,000 viable nucleated cells from a gram of lipoaspirate tissue, between about 500,000 and about 800,000 viable nucleated cells from a gram of lipoaspirate tissue, between about 600,000 and about 1,000,000 viable nucleated cells from a gram of lipoaspirate tissue, or between about 700,000 and about 1,200,000 viable nucleated cells from a gram of lipoaspirate tissue with a processing time of less than 120 minutes, less than 90 minutes, less than 75 minutes, less than 60 minutes, less than 50 minutes, less than 45 minutes, less than 40 minutes, less than 35 minutes, less than 30 minutes, less than 25 minutes, or less than 20 minutes, for at least 70%, at least 75%, at least 80%, at least 85%, or at least 90% of lipoaspirate samples collected using similar liposuction procedures, for example, conventional liposuction.

It is appreciated that with or without further optimization, the system disclosed herein may be capable of recovering at least about 500,000, about 600,000, about 700,000, about 800,000, about 900,000, about 1,000,000, about 1,200,000, about 1,300,000, about 1,500,000, about 1,800,000, about 2,000,000 viable nucleated cells from a gram of lipoaspirate tissue or adipose tissue in average, with average viability of greater than 80%, greater than 85%, greater than 88%, greater than 90%, greater than 92% or greater than 95%. The system disclosed herein may be capable of recovering more than 500,000, more than 600,000, more than 700,000, more than 750,000, more than 800,000, more than 800,000, more than 900,000, more than 999,000, more than 1,000,000, more than 1,100,000, more than 1,200,000, more than 1,250,000, more than 1,300,000, more than 1,500,000, more than 1,750,000, more than 2,000,000, more than 3,000,000, or more than 4,000,000 viable nucleated cells from a gram of adipose tissue. The inter sample coefficient of variance of viable cell recovery using the system disclosed herein may be smaller than about 25%, smaller than about 20%, smaller than about 18%, smaller than about 16%, smaller than about 15%, smaller than about 14%, smaller than about 12%, or smaller than about 10% amongst samples collected from a patient cohort of similar age and body mass index (BMI), using similar liposuction procedures, for example conventional liposuction.

The automated tissue manipulation system disclosed in the present disclosure may be configured to process different amount of sample, for example, about 0.1 g, about 0.2 g, about 0.3 g, about 0.4 g, about 0.5 g, about 0.6 g, about 0.7 g, about 0.8 g, about 0.9 g, about 1 g, about 1.2 g, about 1.5 g, about 1.7 g, about 2 g, about 2.5 g, about 3 g, about 4 g, about 5 g, about 6 g, about 7 g, about 8 g, about 9 g, about 10 g, about 12 g, about 14 g, about 16 g, about 18 g, about 20 g, about 25 g, about 30 g, about 35 g, about 40 g, about 45 g, about 50 g, about 55 g, about 60 g, about 70 g, about 80 g, about 90 g, about 100 g, about 110 g, about 125 g, about 150 g, about 175 g, about 200 g, about 250 g, about 300 g, about 350 g, about 400 g, about 500 g, about 600 g, about 700 g, about 750 g, about 800 g, about 900 g, about 1,000 g, about 1,200 g, about 1,500 g, about 2,000 g, about 0.1 ml, about 0.2 ml, about 0.3 ml, about 0.4 ml, about 0.5 ml, about 0.6 ml, about 0.7 ml, about 0.8 ml, about 0.9 ml, about 1 ml, about 1.2 ml, about 1.5 ml, about 1.7 ml, about 2 ml, about 2.5 ml, about 3 ml, about 4 ml, about 5 ml, about 6 ml, about 7 ml, about 8 ml, about 9 ml, about 10 ml, about 12 ml, about 14 ml, about 16 ml, about 18 ml, about 20 ml, about 25 ml, about 30 ml, about 35 ml, about 40 ml, about 45 ml, about 50 ml, about 55 ml, about 60 ml, about 70 ml, about 80 ml, about 90 ml, about 100 ml, about 110 ml, about 125 ml, about 150 ml, about 175 ml, about 200 ml, about 250 ml, about 300 ml, about 350 ml, about 400 ml, about 500 ml, about 600 ml, about 700 ml, about 750 ml, about 800 ml, about 900 ml, about 1,000 ml, about 1,200 ml, about 1,500 ml, or about 2,000 ml. An automated tissue manipulation system disclosed in the present disclosure may also be configured to process different volume range of sample, for example, between about 0.05 g and about 2,000 g, between about 0.1 g and about 30 g, between about 0.2 g and about 10 g, between about 5 g and about 20 g, between about 1 g and about 30 g, between about 3 g and about 30 g, between about 20 g and about 60 g, between about 10 g and about 50 g, between about 10 ml and about 100 ml, between about 20 ml and about 75 ml, between about 30 ml and about 60 ml, between about 20 ml and about 50 ml, between about 40 ml and about 60 ml, between about 50 ml and about 200 ml, between about 100 ml and about 900 ml, between about 50 ml and about 500 ml, between about 200 ml and about 2,000 ml, between about 15 ml and about 60 ml, between about 500 ml and about 1,000 ml, between about 100 ml and about 600 ml, between about 5 ml and about 80 ml, etc. The system may be configured to process a sample in about 5 minutes, about 10 minutes, about 12 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 75 minutes, about 80 minutes, about 90 minutes, about 100 minutes, about 105 minutes, about 120 minutes, about 135 minutes, about 150 minutes, about 180 minutes, about 200 minutes, about 210 minutes, about 4 hours, about 5 hours, about 6 hours, about 7 hours, about 8 hours, about 10 hours, about 12 hours, about 18 hours, or about 24 hours. The system may further be configured to have an output volume of about 10 ml, about 15 ml, about 20 ml, about 25 ml, about 30 ml, about 35 ml, about 40 ml, about 45 ml, about 48 ml, about 50 ml, about 52 ml, about 55 ml, about 58 ml, about 59 ml, or about 60 ml.

### Example 3

### Washing and Dispensing Lipoaspirate

A tissue manipulation system as shown in FIG. 2E, comprising a processing unit including a mesh filter of 125 μm pore size, an output tissue pump including four duckbill valves and two 10 ml syringes (as shown in FIG. 3C), a cannula hand piece comprising a button, a modulator comprising a flexible conduit of about 3.2 mm inner diameter and a duckbill valve, and a 16 gauge, 10 cm long injection cannula was used to wash and dispense lipoaspirate. A lipoaspirate sample is injected into the processing unit, and washed with lactated Ringer's solution. The output tissue pump is configured to pump about 100 μl of lipoaspirate towards the modulator. The modulator is actuated by squeezing the flexible conduit using the button on the cannula hand piece.

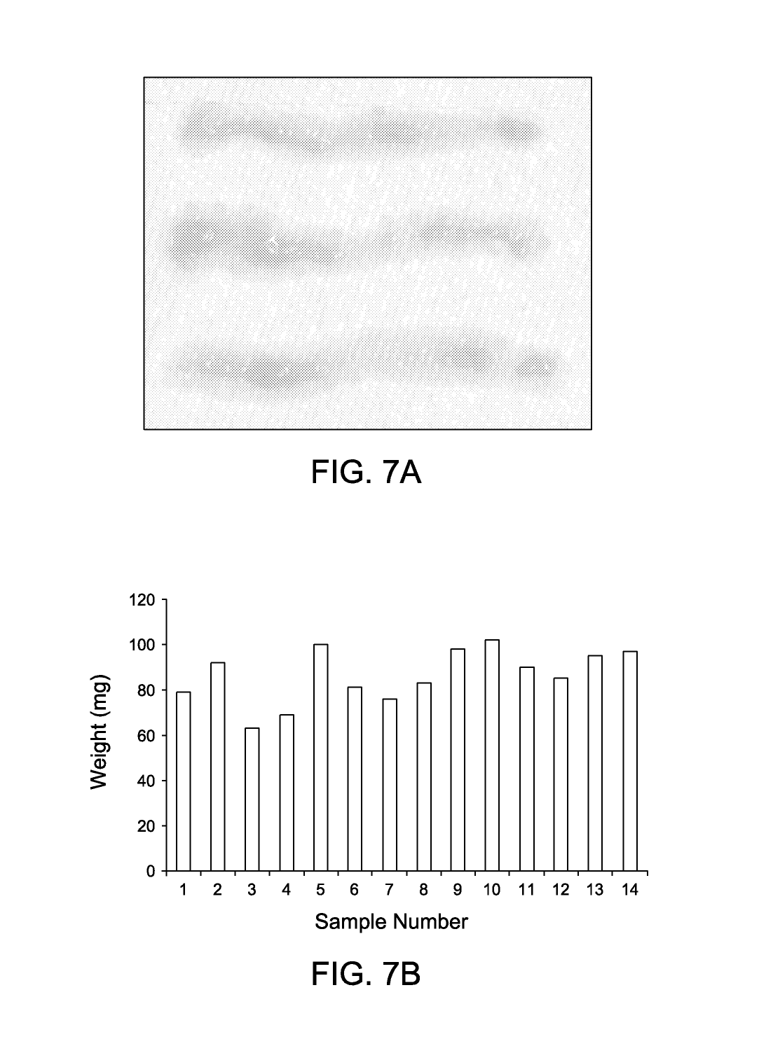

FIG. 7A shows three lines of lipoaspirate drawn on a piece of tissue paper using the injection cannula of the system. Each line represents a discrete volume of lipoaspirate, and the volume is pre-set to be about 100 μl nominally. It can be seen that the three lines contain about the same volume, even though each line includes pieces of adipose tissues of different sizes. In the next demonstration, fourteen discrete volumes of lipoaspirate is dispensed and weighed to measure the weight of each bit (also referred to as “discrete volume”), with the nominal volume set to be about 100 μl per tissue bit. The results are shown in FIG. 7B. The average weight of a bit is about 86.4 g, and the standard deviation of the weights of the bits is about 11.9 g, corresponding to a coefficient of variance on the bit weight of about 14%. This level of consistency and coefficient of variance of lipoaspirate dispensing may be very difficult to achieve using a manually controlled syringe.

It is appreciated that the tissue manipulation system disclosed in the present disclosure may be capable of dispensing tissue bits of about 10 μl, about 15 μl, about 20 μl, about 25 μl, about 30 μl, about 40 μl, about 50 μl, about 60 μl, about 70 μl, about 80 μl, about 100 μl, about 120 μl, about 150 μl, about 175 μl, about 200 μl, about 250 μl, about 300 μl, about 400 μl, about 500 piper bit, with a consistency of coefficient of variance of smaller than about 30%, about 25%, about 20%, about 15%, about 14%, about 13%, about 12%, about 11%, about 10%, about 9%, about 8%, about 7%, about 6%, about 5%, about 4%, or about 3%.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of this disclosure. It should be understood that any portion of any embodiment disclosed herein may be included in any other embodiment or substituted for any other portion of any other embodiment. Accordingly, the foregoing description and drawings are by way of example only, and the scope of the aspects and embodiments disclosed herein should be determined from proper construction of the appended claims, and their equivalents.

1. A system for manipulation of a tissue sample, the system comprising:
a chassis;
a chamber defined in the chassis and configured to receive and retain a complementary device including a flexible sample processing compartment selectively fluidly connected to a source of a first solution, and a waste chamber selectively fluidly connected to an outlet of the sample processing compartment, the complementary device configured to retain the tissue sample and receive the first solution during manipulation of the tissue sample by the system;
a fluid mixing sub-system disposed in the chamber and configured to agitate and mix a fluid including the first solution and the tissue sample within the sample processing compartment;
a temperature control sub-system including at least one of a first heating element and a first cooling element configured and arranged to be in thermal communication with the sample processing compartment; and
an electronic controller in communication with, and programmed to control operation of, the fluid mixing sub-system and the temperature control sub-system.

2. The system of claim 1, further comprising:
a fluid control sub-system disposed in the chassis and controlled by the electronic controller; and
a user interface in communication with the electronic controller.

3. The system of claim 1, wherein the waste chamber and sample processing compartment are disposed between sheets of a flexible material.

4. The system of claim 1, wherein the sample processing compartment and the waste chamber are disposed between common sheets of a flexible material.

5. The system of claim 1, wherein the fluid mixing sub-system is configured to manipulate at least a part of the flexible sample processing compartment, providing massaging action to the flexible sample processing compartment.

6. The system of claim 2, wherein the fluid control sub-system includes a valve actuator configured to mechanically manipulate a valve disposed in the complementary device, the valve having a state providing for gravity drain of a fluid from the sample processing compartment into the waste chamber.

7. The system of claim 2, wherein the fluid control sub-system further includes a first pump configured to withdraw the first solution and direct the first solution into the sample processing compartment.

8. The system of claim 7, wherein the first pump comprises a first syringe included in the complementary device and the fluid control sub-system further includes a first linear actuator configured to manipulate a plunger of the first syringe.

9. The system of claim 7, wherein the system further includes a second pump configured to direct a second solution into the sample processing compartment.

10. The system of claim 9, wherein the second pump comprises a second syringe included in the complementary device and the fluid control sub-system further includes a second linear actuator configured to manipulate a plunger of the second syringe.

11. The system of claim 9, wherein the first solution is a rinse solution and the second solution is a reagent solution comprising enzyme.

12. The system of claim 2, further comprising a third syringe configured to withdraw treated cells from the complementary device.

13. The system of claim 12, wherein the fluid control sub-system further includes a third linear actuator configured to manipulate a plunger of the third syringe.

14. The system of claim 1, further comprising a detection feedback system including a sensor in communication with the electronic controller, the sensor configured and arranged to one of provide an indication of whether the complementary device is properly mounted within the chamber, provide an indication of whether a syringe is properly mounted on the system, provide an indication of whether a door of the chamber is closed, and provide an indication of whether the door of the chamber is locked.

15. The system of claim 1, further comprising a detection feedback system including a sensor in communication with the electronic controller, the sensor configured and arranged to provide an indication of a weight of a bag of a rinsing solution disposed on a platform coupled to the chassis, the fluid control sub-system configured to dispense an volume of rinsing solution into the sample processing compartment determined by a change in weight of the bag.

16. The system of claim 1, further comprising an identification tag reader configured to read an identification tag included on the complementary device.

17. The system of claim 16, wherein the controller is configured to execute a tissue manipulation protocol defined by information read from the identification tag by the identification tag reader.

18. The system of claim 1, wherein the temperature control sub-system is configured to be in thermal communication with the sample processing compartment using forced air.

19. The system of claim 1, wherein the temperature control sub-system includes a plate configured to be in thermal communication with the at least one of the first heating element and the first cooling element and in physical contact with the complementary device.

20. The system of claim 1, wherein the fluid mixing sub-system includes a roller configured to agitate and mix fluid within the sample processing compartment.

21. The system of claim 1, wherein the fluid mixing sub-system includes a rotating arm configured to agitate and mix fluid within the sample processing compartment.

22. The system of claim 1, wherein the fluid mixing sub-system includes a moving plate configured to agitate and mix fluid within the sample processing compartment.

23. The system of claim 1, wherein the complementary device further includes a filter configured to remove debris from treated cells.

24. The system of claim 1, wherein the sample processing compartment has a surface to volume ratio of greater than 3 cm⁻¹.

25. The system of claim 2, wherein the fluid control system further includes a sensor in communication with the electronic controller, the sensor configured to monitor one of a flow rate and a property of a fluid in the system selected from a color of the tissue sample and a turbidity of the tissue sample.

26. The system of claim 1, wherein the temperature control sub-system is configured to heat the tissue in the sample processing compartment to 35° C. or greater within 2 minutes.

27. A method of processing a tissue sample, the method comprising:
mounting a device including a sample processing compartment disposed between sheets of a flexible material, and a waste chamber selectively fluidly connected to an outlet of the sample processing chamber, onto a processing chamber of a tissue manipulation apparatus;
introducing the tissue sample into the sample processing compartment of the device;
introducing a fluid into the sample processing compartment to treat the tissue;
agitating and mixing the tissue sample within the sample processing compartment with a fluid mixing sub-system disposed at the processing chamber under control of an electronic controller of the tissue manipulation apparatus; and
one of heating and cooling the tissue sample with a temperature control sub-system including at least one of a first heating element and a first cooling element disposed at the processing chamber and in thermal communication with the sample processing compartment under control of the electronic controller.

28. The method of claim 27, further comprising washing the tissue sample in the sample processing compartment by dispensing a measured volume of a rinse solution into the sample processing compartment under control of the electronic controller.

29. The method of claim 27, further comprising digesting the tissue sample in the sample processing compartment by dispensing a measured volume of a dissociation solution into the sample processing compartment under control of the electronic controller.

30. The method of claim 29, wherein the dissociation solution contains enzyme.

31. The method of claim 27, wherein the sample processing compartment and the waste chamber are disposed between common sheets of a flexible material.

32. The method of claim 27, further comprising mechanically manipulating a valve in fluid communication between the sample processing compartment and the waste chamber under control of the electronic controller, mechanically manipulating the valve causing a waste fluid to flow under the influence of gravity from the sample processing compartment to the waste chamber.

33. The method of claim 27, further comprising withdrawing a fluid containing cells from the device under control of the electronic controller.

34. The method of claim 29, further comprising removing debris using a filter included in the device.

35. The method of claim 27, wherein the tissue is an adipose tissue having a weight, wherein the method further comprises digesting the tissue sample in the sample processing compartment by dispensing a measured volume of a dissociation solution comprising collagenase into the sample processing compartment under control of the electronic controller, and wherein the method further comprises collecting a fluid containing viable nucleated cells from the device.

36. The method of claim 35, wherein the number of viable nucleated cells collected from a unit weight of the adipose tissue is more than 700,000 per gram of adipose tissue.

37. The method of claim 35, wherein the intra-sample coefficient of variance of viable nucleated cells collected from a unit weight of the adipose tissue is no greater than 5%.

38. The method of claim 35, wherein the method is performed in a time of no longer than 55 minutes.
